(19) United States
(12) Patent Application Publication
(10) Pub. No.: US 2023/0233677 A1
(43) Pub. Date: Jul. 27, 2023

(54) COMPOSITIONS AND METHODS FOR ENHANCING IMMUNE RESPONSE

(71) Applicant: HANGZHOU LEADING EDGE PHARMACEUTICAL LTD., Hangzhou, Zhejiang (CN)

(72) Inventors: Meng XU, Hangzhou (CN); Xuanming YANG, Hangzhou (CN); Wenxin DONG, Hangzhou (CN)

(73) Assignee: HANGZHOU LEADING EDGE PHARMACEUTICAL LTD., Hangzhou, Zhejiang (CN)

(21) Appl. No.: 18/001,372

(22) PCT Filed: Jun. 8, 2021
(86) PCT No.: PCT/CN2021/098870

§ 371 Date: Dec. 9, 2022

(30) Foreign Application Priority Data
Jun. 9, 2020 (WO) PCT/CN2020/095211

(51) Int. Cl. A61K 39/00; A61K 39/395; C07K 16/18; C12N 15/113; A61P 35/00; C12N 5/0783

(52) U.S. Cl. CPC A61K 39/4611; A61K 39/3955; C07K 16/18; C12N 15/113; A61P 35/00; C12N 5/0636; C12N 2310/16; C12N 2310/14; C12N 2310/531; C12N 2501/2302; C12N 2501/2321

(57) ABSTRACT

A modified immune cell that has attenuated expression and/or activity of YTH N6-Methyladenosine RNA Binding Protein 2 (YTHDF2), and enhanced anti-tumor activity. A composition for stimulating T cell-mediated immune response to a cancer cell and/or a tumor antigen, including an agent capable of attenuating the expression and/or activity of YTHDF2, and a pharmaceutically acceptable excipient. A composition for treating cancer, comprising an agent capable of attenuating the expression and/or activity of YTHDF2. A method for activating an immune cell. A method for generating an immune cell. A method for treating a disease, disorder or condition associated with an expression of a tumor antigen in a subject in need thereof. A method for stimulating a T cell-mediated immune response to a cancer cell and/or a tumor antigen in a subject in need thereof.

### BACKGROUND

Spontaneous T cell infiltration and proliferation in tumor microenvironment is critical for the clinical efficacy of immunotherapies. However, in many patients, tumor infiltrating T cells cannot survive or provide lasting T cell response required for complete tumor rejection. Identifying molecular pathways that influence the dysfunctional state of tumor infiltrating T cells could provide targets for improving the response to immunotherapy.

Although significant advances have been made in the field of cancer immunotherapy, yet, more potent and more effective therapies are still in urgent need.

### SUMMARY OF THE PRESENT APPLICATION

The present application relates to compositions and methods for enhancing immune response, such as immune responses to cancer cells and/or tumor antigens. In one aspect, the present inventors found that the protein YTH N6-Methyladenosine RNA Binding Protein 2 (YTHDF2) is associated with the expression of T cell exhaustion signature genes. Mice lacking YTHDF2 in T cells demonstrated better anti-tumor immunity for lymphoma and for solid tumors (such as melanoma and colon cancer). Functions of tumor-infiltrating T cell were enhanced in YTHDF2-deficient mice. Furthermore, the divergence of T cell exhaustion was rescued toward a fate of memory-like or stem-like CD8⁺ T cell.

In one aspect, the present application provides a modified immune cell. Comparing to an unmodified corresponding immune cell, the modified immune cell has attenuated expression and/or activity of YTHDF2, and enhanced anti-tumor activity.

In some embodiments, the immune cell is an immune effector cell.

In some embodiments, the immune cell is a T cell. In some embodiments, the immune cell is a CD4⁺ cell. In some embodiments, the immune cell is a CD8⁺ cell. In some embodiments, the immune cell is a tumor infiltrating T cell.

In some embodiments, the immune cell is a cell (e.g., a population of cells, such as a population of immune effector cells) engineered to express a chimeric antigen receptor (CAR). In some embodiments, the immune cell is a CAR-T cell.

In some embodiments, the CAR used in the present application comprises an antigen-binding domain, a transmembrane domain, and an intracellular signaling domain. In some embodiments, the antigen-binding domain binds to a tumor antigen (e.g., CD20 or CLDN18.2). In some embodiments, the antigen is CD20, Claudin protein, CLDN18 or CLDN18.2. In some embodiments, the antigen-binding domain is an antibody or antibody fragment derived from Rituximab. In some embodiments, the transmembrane domain of said CAR comprises: (i) an amino acid sequence having at least one, two or three modifications but not more than 20, 10 or 5 modifications of an amino acid sequence of SEQ ID NO: 10, or a sequence with 95-100 (e.g., 95-96, 95-97, 95-98, 95-99, 95-99.5 or more) % identity to an amino acid sequence of SEQ ID NO: 10; or (ii) the sequence of SEQ ID NO: 10. In some embodiments, the antigen binding domain of the CAR is connected to the transmembrane domain by a hinge region, wherein said hinge region comprises SEQ ID NO: 9, or a sequence with 95-100 (e.g., 95-96, 95-97, 95-98, 95-99, 95-99.5 or more) % identity thereof. In some embodiments, the intracellular signaling domain of said CAR comprises a primary signaling domain and/or a costimulatory signaling domain, wherein the primary signaling domain comprises a functional signaling domain of CD3 zeta. In some embodiments, the primary signaling domain of said CAR comprises: (i) an amino acid sequence having at least one, two or three modifications but not more than 20, 10 or 5 modifications of an amino acid sequence of SEQ ID NO: 8, or a sequence with 95-100 (e.g., 95-96, 95-97, 95-98, 95-99, 95-99.5 or more) % identity to an amino acid sequence of SEQ ID NO: 8; or (ii) the amino acid sequence of SEQ ID NO: 8. In some embodiments, the intracellular signaling domain of said CAR comprises a costimulatory signaling domain, or a primary signaling domain and a costimulatory signaling domain, wherein the costimulatory signaling domain comprises a functional signaling domain of 4-1BB (CD137). In some embodiments, the costimulatory signaling domain of said CAR comprises an amino acid sequence having at least one, two or three modifications but not more than 20, 10 or 5 modifications of an amino acid sequence of SEQ ID NO: 7, or a sequence with 95-100 (e.g., 95-96, 95-97, 95-98, 95-99, 95-99.5 or more) % identity to an amino acid sequence of SEQ ID NO: 7. In some embodiments, the intracellular domain of said CAR comprises the sequence of SEQ ID NO: 7, and the sequence of SEQ ID NO: 8, wherein the sequences comprising the intracellular signaling domain are expressed in the same open reading frame and as a single polypeptide chain.

In some embodiments, the immune cell is a cell (e.g., a population of cells, such as a population of immune effector cells) engineered to express a T cell receptor. In some embodiments, the immune cell is a TCR-T cell.

In some embodiments, the unmodified corresponding immune cell is TCF1⁻.

In some embodiments, the unmodified corresponding immune cell is Tim3⁺.

In some embodiments, the unmodified corresponding immune cell is PD-1⁺.

In some embodiments, the modified immune cell is PD-1⁺ or PD-1⁻.

In some embodiments, the modified immune cell is TCF1⁺ and/or TCF7⁺.

In some embodiments, the modified immune cell is Tim3⁻.

In some embodiments, the immune cell has been modified with an agent capable of attenuating the expression and/or activity of YTHDF2. In some embodiments, the immune cells (e.g., engineered or modified immune effector cells, e.g., T cells) of the present application comprise an agent capable of attenuating the expression and/or activity of YTHDF2.

In some embodiments, the agent capable of attenuating the expression and/or activity of YTHDF2 comprises an agent capable of attenuating the expression and/or activity of a gene encoding YTHDF2, and/or an agent capable of attenuating the expression and/or activity of the YTHDF2 protein.

In some embodiments, the immune cells are human cells.

In some embodiments, the agent capable of attenuating the expression and/or activity of YTHDF2 comprises one or more of the following: a macromolecule and a small molecule.

In some embodiments, the agent capable of attenuating the expression and/or activity of YTHDF2 comprises one or more of the following: a polypeptide and a nucleic acid molecule.

In some embodiments, the agent capable of attenuating the expression and/or activity of YTHDF2 comprises one or more of the following: an antibody or a derivative thereof, an antibody-drug conjugate, a fusion protein, and an antisense molecule.

In some embodiments, the agent capable of attenuating the expression and/or activity of YTHDF2 comprises one or more of the following: a ubiquitin, a PROTAC, a dsRNA, a siRNA, a shRNA, an aptamer, and a gRNA.

In some embodiments, the agent capable of attenuating the expression and/or activity of YTHDF2 comprises one or more of the following: a mutant or a variant of YTHDF2 protein capable of attenuating the activity of an endogenous YTHDF2; and a nucleic acid molecule encoding for the mutant or variant of YTHDF2 protein. In some embodiments, the agent capable of attenuating the expression and/or activity of YTHDF2 comprises a dominant negative (e.g., incapable of recognizing, binding to, and/or modifying m⁶A RNA) YTHDF2, or nucleic acid encoding said dominant negative YTHDF2.

In some embodiments, the immune cell has been subjected to a modification that results in a complete or partial deletion, a complete or partial replacement and/or reduced expression of a gene expressing YTHDF2.

In some embodiments, the agent capable of attenuating the expression and/or activity of YTHDF2 is (1) a gene editing system targeted to one or more sites within the gene encoding YTHDF2, or its regulatory elements, e.g., Ythdf2, or its regulatory elements; (2) nucleic acid encoding one or more components of said gene editing system; or (3) combinations thereof.

In some embodiments, the gene editing system is selected from the group consisting of: a CRISPR/Cas9 system, a zinc finger nuclease system, a TALEN system and a meganuclease system.

In some embodiments, the gene editing system is a CRISPR/Cas system comprising a gRNA molecule comprising a targeting sequence which hybridize to a target sequence of a Ythdf2 gene. In some embodiments, the gene editing system binds to a target sequence in an early exon or intron of a gene encoding YTHDF2. In some embodiments, the gene editing system binds to a target sequence upstream of exon 4, e.g., in exon 1, exon 2, and/or exon 3 of a gene encoding YTHDF2.

In some embodiments, the gene editing system binds to a target sequence in a late exon or intron of a gene encoding YTHDF2. In some embodiments, the gene editing system binds to a target sequence downstream of exon 3, e.g., in exon 4, exon 5, exon 6, exon 7, and/or exon 8 of a gene encoding YTHDF2.

In some embodiments, the gene editing system binds to a target sequence in exon 1, exon 2, exon 3, exon 4, exon 5, exon 6, exon 7, and/or exon 8 of a gene encoding YTHDF2.

In some embodiments, the targeting sequence is a targeting sequence as set forth in SEQ ID NO. 17.

In some embodiments, the agent capable of attenuating the expression and/or activity of YTHDF2 is an siRNA or shRNA specific for Ythdf2, or nucleic acid encoding said siRNA or shRNA. In some embodiments, the siRNA or shRNA comprises a sequence complementary to a sequence of a Ythdf2 mRNA.

In one aspect, the present application provides a composition, comprising a modified immune cell of the present application, and optionally a pharmaceutically acceptable excipient.

In one aspect, the present application provides a composition for stimulating T cell-mediated immune response to a cancer cell and/or a tumor antigen, comprising an agent capable of attenuating the expression and/or activity of YTHDF2, and optionally a pharmaceutically acceptable excipient.

In one aspect, the present application provides a composition for treating cancer, comprising an agent capable of attenuating the expression and/or activity of YTHDF2, and optionally a pharmaceutically acceptable excipient.

In some embodiments of the composition of the present application, the agent capable of attenuating the expression and/or activity of YTHDF2 comprises an agent capable of attenuating the expression and/or activity of a gene encoding YTHDF2, and/or an agent capable of attenuating the expression and/or activity of the YTHDF2 protein.

In some embodiments of the composition of the present application, the agent capable of attenuating the expression and/or activity of YTHDF2 comprises one or more of the following: a macromolecule and a small molecule.

In some embodiments of the composition of the present application, the agent capable of attenuating the expression and/or activity of YTHDF2 comprises one or more of the following: a polypeptide and a nucleic acid molecule.

In some embodiments of the composition of the present application, the agent capable of attenuating the expression and/or activity of YTHDF2 comprises one or more of the following: an antibody or a derivative thereof, an antibody-drug conjugate, a fusion protein, and an antisense molecule.

In some embodiments of the composition of the present application, the agent capable of attenuating the expression and/or activity of YTHDF2 comprises one or more of the following: a ubiquitin, a PROTAC, a dsRNA, a siRNA, a shRNA, an aptamer, and a gRNA.

In some embodiments of the composition of the present application, the agent capable of attenuating the expression and/or activity of YTHDF2 comprises one or more of the following: a mutant or a variant of YTHDF2 protein capable of attenuating the activity of an endogenous YTHDF2; and a nucleic acid molecule encoding for the mutant or variant of YTHDF2 protein. In some embodiments of the composition of the present application, the agent capable of attenuating the expression and/or activity of YTHDF2 comprises a dominant negative (e.g., incapable of recognizing, binding to, and/or modifying m⁶A RNA) YTHDF2, or nucleic acid encoding said dominant negative YTHDF2.

In some embodiments of the composition of the present application, the agent capable of attenuating the expression and/or activity of YTHDF2 is (1) a gene editing system targeted to one or more sites within the gene encoding YTHDF2, or its regulatory elements, e.g., Ythdf2, or its regulatory elements; (2) nucleic acid encoding one or more components of said gene editing system; or (3) combinations thereof.

In some embodiments of the composition of the present application, the gene editing system is selected from the group consisting of: a CRISPR/Cas9 system, a zinc finger nuclease system, a TALEN system and a meganuclease system.

In some embodiments of the composition of the present application, the gene editing system is a CRISPR/Cas system comprising a gRNA molecule comprising a targeting sequence which hybridize to a target sequence of a Ythdf2 gene. In some embodiments of the composition of the present application, the gene editing system binds to a target sequence in an early exon or intron of a gene encoding YTHDF2. In some embodiments of the composition of the present application, the gene editing system binds to a target sequence upstream of exon 4, e.g., in exon 1, exon 2, and/or exon 3 of a gene encoding YTHDF2.

In some embodiments of the composition of the present application, the gene editing system binds to a target sequence in a late exon or intron of a gene encoding YTHDF2. In some embodiments of the composition of the present application, the gene editing system binds to a target sequence downstream of exon 3, e.g., in exon 4, exon 5, exon 6, exon 7, and/or exon 8 of a gene encoding YTHDF2.

In some embodiments, the gene editing system binds to a target sequence in exon 1, exon 2, exon 3, exon 4, exon 5, exon 6, exon 7, and/or exon 8 of a gene encoding YTHDF2.

In some embodiments of the composition of the present application, the targeting sequence is a targeting sequence as set forth in SEQ ID NO.17.

In some embodiments of the composition of the present application, the agent capable of attenuating the expression and/or activity of YTHDF2 is an siRNA or shRNA specific for Ythdf2, or nucleic acid encoding said siRNA or shRNA. In some embodiments of the composition of the present application, the siRNA or shRNA comprises a sequence complementary to a sequence of a Ythdf2 mRNA.

In some embodiments, the composition of the present application further comprises a second active ingredient. In some embodiments, the second active ingredient is an anti-cancer agent. In some embodiments, the second active ingredient comprises a cancer immunotherapy. In some embodiments, the second active ingredient comprises an immune checkpoint inhibitor. In some embodiments, the second active ingredient comprises an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO inhibitor. In some embodiments, the second active ingredient comprises pembrolizumab, nivolumab, cemiplimab, atezolizumab, avelumab, durvalumab, and/or ipilimumab.

In some embodiments of the composition of the present application, the second active ingredient is comprised in a separate container and is not mixed with the modified immune cell, or with the agent capable of attenuating the expression and/or activity of YTHDF2.

In one aspect, the present application provides a method for activating an immune cell, the method comprises attenuating an expression and/or activity of YTHDF2 in the immune cell.

In one aspect, the present application provides a method for generating an immune cell having enhanced anti-tumor activity, the method comprises attenuating an expression and/or activity of YTHDF2 in the immune cells.

In one aspect, the present application provides a method for preventing and/or reversing exhaustion of an immune cell, the method comprises attenuating an expression and/or activity of YTHDF2 in the immune cells.

In some embodiments of a method of the present application, the immune cell is an immune effector cell. In some embodiments of a method of the present application, the immune cell is a T cell. In some embodiments of a method of the present application, the immune cell is a CD4⁺ cell. In some embodiments of a method of the present application, the immune cell is a CD8⁺ cell. In some embodiments of a method of the present application, the immune cell is a tumor infiltrating T cell.

In some embodiments of a method of the present application, the immune cell is a cell (e.g., a population of cells, such as a population of immune effector cells) engineered to express a chimeric antigen receptor (CAR). In some embodiments, the immune cell is a CAR-T cell.

In some embodiments of a method of the present application, the CAR used in the present application comprises an antigen-binding domain, a transmembrane domain, and an intracellular signaling domain. In some embodiments, the antigen-binding domain binds to a tumor antigen (e.g., CD20 or CLDN18.2). In some embodiments, the antigen is CD20 or CLDN18.2. In some embodiments, the antigen-binding domain is an antibody or antibody fragment derived from Rituximab. In some embodiments, the transmembrane domain of said CAR comprises: (i) an amino acid sequence having at least one, two or three modifications but not more than 20, 10 or 5 modifications of an amino acid sequence of SEQ ID NO: 10, or a sequence with 95-100 (e.g., 95-96, 95-97, 95-98, 95-99, 95-99.5 or more) % identity to an amino acid sequence of SEQ ID NO: 10; or (ii) the sequence of SEQ ID NO: 10. In some embodiments, the antigen binding domain of the CAR is connected to the transmembrane domain by a hinge region, wherein said hinge region comprises SEQ ID NO: 9, or a sequence with 95-100 (e.g., 95-96, 95-97, 95-98, 95-99, 95-99.5 or more) % identity thereof. In some embodiments, the intracellular signaling domain of said CAR comprises a primary signaling domain and/or a costimulatory signaling domain, wherein the primary signaling domain comprises a functional signaling domain of CD3 zeta. In some embodiments, the primary signaling domain of said CAR comprises: (i) an amino acid sequence having at least one, two or three modifications but not more than 20, 10 or 5 modifications of an amino acid sequence of SEQ ID NO: 8, or a sequence with 95-100 (e.g., 95-96, 95-97, 95-98, 95-99, 95-99.5 or more) % identity to an amino acid sequence of SEQ ID NO: 8; or (ii) the amino acid sequence of SEQ ID NO: 8. In some embodiments, the intracellular signaling domain of said CAR comprises a costimulatory signaling domain, or a primary signaling domain and a costimulatory signaling domain, wherein the costimulatory signaling domain comprises a functional signaling domain of 4-1BB (CD137). In some embodiments, the costimulatory signaling domain of said CAR comprises an amino acid sequence having at least one, two or three modifications but not more than 20, 10 or 5 modifications of an amino acid sequence of SEQ ID NO: 7, or a sequence with 95-100 (e.g., 95-96, 95-97, 95-98, 95-99, 95-99.5 or more) % identity to an amino acid sequence of SEQ ID NO: 7. In some embodiments, the intracellular domain of said CAR comprises the sequence of SEQ ID NO: 7, and the sequence of SEQ ID NO: 8, wherein the sequences comprising the intracellular signaling domain are expressed in the same open reading frame and as a single polypeptide chain.

In some embodiments of a method of the present application, the immune cell is a cell (e.g., a population of cells, such as a population of immune effector cells) engineered to express a T cell receptor. In some embodiments, the immune cell is a TCR-T cell.

In some embodiments of a method of the present application, the attenuating comprises modifying the immune cell with an agent capable of attenuating the expression and/or activity of YTHDF2 in the immune cell.

In some embodiments of a method of the present application, the agent capable of attenuating the expression and/or activity of YTHDF2 comprises an agent capable of attenuating the expression and/or activity of a gene encoding YTHDF2, and/or an agent capable of attenuating the expression and/or activity of the YTHDF2 protein.

In some embodiments of a method of the present application, the agent capable of attenuating the expression and/or activity of YTHDF2 comprises one or more of the following: a macromolecule and a small molecule.

In some embodiments of a method of the present application, the agent capable of attenuating the expression and/or activity of YTHDF2 comprises one or more of the following: a polypeptide and a nucleic acid molecule.

In some embodiments of a method of the present application, the agent capable of attenuating the expression and/or activity of YTHDF2 comprises one or more of the following: an antibody or a derivative thereof, an antibody-drug conjugate, a fusion protein, and an antisense molecule.

In some embodiments of a method of the present application, the agent capable of attenuating the expression and/or activity of YTHDF2 comprises one or more of the following: a ubiquitin, a PROTAC, a dsRNA, a siRNA, a shRNA, an aptamer, and a gRNA.

In some embodiments of a method of the present application, the agent capable of attenuating the expression and/or activity of YTHDF2 comprises one or more of the following: a mutant or a variant of YTHDF2 protein capable of attenuating the activity of an endogenous YTHDF2; and a nucleic acid molecule encoding for the mutant or variant of YTHDF2 protein. In some embodiments of the composition of the present application, the agent capable of attenuating the expression and/or activity of YTHDF2 comprises a dominant negative (e.g., incapable of recognizing, binding to, and/or modifying m⁶A RNA) YTHDF2, or nucleic acid encoding said dominant negative YTHDF2.

In some embodiments of a method of the present application, the agent capable of attenuating the expression and/or activity of YTHDF2 is (1) a gene editing system targeted to one or more sites within the gene encoding YTHDF2, or its regulatory elements, e.g., Ythdf2, or its regulatory elements; (2) nucleic acid encoding one or more components of said gene editing system; or (3) combinations thereof.

In some embodiments of a method of the present application, the gene editing system is selected from the group consisting of: a CRISPR/Cas9 system, a zinc finger nuclease system, a TALEN system and a meganuclease system.

In some embodiments of a method of the present application, the gene editing system is a CRISPR/Cas system comprising a gRNA molecule comprising a targeting sequence which hybridize to a target sequence of a Ythdf2 gene. In some embodiments of the composition of the present application, the gene editing system binds to a target sequence in an early exon or intron of a gene encoding YTHDF2. In some embodiments of the composition of the present application, the gene editing system binds to a target sequence upstream of exon 4, e.g., in exon 1, exon 2, and/or exon 3 of a gene encoding YTHDF2.

In some embodiments of a method of the present application, the gene editing system binds to a target sequence in a late exon or intron of a gene encoding YTHDF2. In some embodiments of the composition of the present application, the gene editing system binds to a target sequence downstream of exon 3, e.g., in exon 4, exon 5, exon 6, exon 7, and/or exon 8 of a gene encoding YTHDF2.

In some embodiments of a method of the present application, the gene editing system binds to a target sequence in exon 1, exon 2, exon 3, exon 4, exon 5, exon 6, exon 7, and/or exon 8 of a gene encoding YTHDF2.

In some embodiments of a method of the present application, the targeting sequence is a targeting sequence as set forth in SEQ ID NO.17.

In some embodiments of a method of the present application, the agent capable of attenuating the expression and/or activity of YTHDF2 is an siRNA or shRNA specific for Ythdf2, or nucleic acid encoding said siRNA or shRNA. In some embodiments of the composition of the present application, the siRNA or shRNA comprises a sequence complementary to a sequence of a Ythdf2 mRNA.

In some embodiments of a method of the present application, the attenuating comprises subjecting the immune cell to a modification that results in a complete or partial deletion, a complete or partial replacement and/or reduced expression of a gene expressing YTHDF2.

In some embodiments, the method is an in vivo method, an in vitro method, and/or an ex vivo method.

In one aspect, the present application provides a method for treating a disease, disorder or condition associated with an expression of a tumor antigen in a subject in need thereof, comprising administering to the subject: an agent capable of attenuating an expression and/or activity of YTHDF2; and/or a modified immune cell of the present application.

In some embodiments, the disease, disorder or condition is cancer. In some embodiments, the cancer is selected from the group consisting of a hematological cancer, a lymphoma, and a solid tumor. In some embodiments, the cancer is selected from the group consisting of melanoma, colon cancer, pancreatic cancer, breast cancer, lung cancer, and liver cancer.

In one aspect, the present application provides a method for treating cancer in a subject in need thereof, comprising administering to the subject: an agent capable of attenuating an expression and/or activity of YTHDF2; and/or a modified immune cell of the present application. In some embodiments, the cancer is selected from the group consisting of a hematological cancer, a lymphoma, and a solid tumor. In some embodiments, the cancer is selected from the group consisting of melanoma, colon cancer, pancreatic cancer, breast cancer, lung cancer, and liver cancer.

In one aspect, the present application provides a method for stimulating a T cell-mediated immune response to a cancer cell and/or a tumor antigen in a subject in need thereof, comprising administering to the subject: an agent capable of attenuating an expression and/or activity of YTHDF2; and/or a modified immune cell of the present application.

In one aspect, the present application provides a method for providing an anti-tumor immunity in a subject in need thereof, comprising administering to the subject: an agent capable of attenuating an expression and/or activity of YTHDF2; and/or a modified immune cell of the present application.

In one aspect, the present application provides a method for preventing and/or reversing exhaustion of T cells in a subject in need thereof, comprising administering to the subject: an agent capable of attenuating an expression and/or activity of YTHDF2; and/or a modified immune cell of the present application. In some embodiments, the exhaustion of T cells is exhaustion of CD8⁺ T cells.

In some embodiments of a method of the present application, the subject is a cancer patient. In some embodiments, the subject is a patient of a cancer selected from the group consisting of a hematological cancer, a lymphoma, and a solid tumor. In some embodiments, the subject is a patient of a cancer selected from the group consisting of melanoma, colon cancer, pancreatic cancer, breast cancer, lung cancer, and liver cancer.

In some embodiments of a method of the present application, the subject has received, is receiving, and/or will receive an additional therapy, such as an anti-cancer treatment. In some embodiments, the anti-cancer treatment comprises a cancer immunotherapy. In some embodiments, the anti-cancer treatment comprises or is an immune checkpoint inhibitor. In some embodiments, the anti-cancer treatment comprises an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO inhibitor. In some embodiments, the anti-cancer treatment comprises pembrolizumab, nivolumab, cemiplimab, atezolizumab, avelumab, durvalumab, and/or ipilimumab.

In some embodiments, the method further comprises administering to the subject one or more additional anti-cancer treatment. In some embodiments, the additional anti-cancer treatment comprises a cancer immunotherapy. In some embodiments, the additional anti-cancer treatment comprises an immune checkpoint inhibitor. In some embodiments, the additional anti-cancer treatment comprises an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO inhibitor. In some embodiments, the additional anti-cancer treatment comprises pembrolizumab, nivolumab, cemiplimab, atezolizumab, avelumab, durvalumab, and/or ipilimumab.

In one aspect, the present application provides use of an agent capable of attenuating an expression and/or activity of YTHDF2 in the manufacture of a composition and/or of a medicament for one or more of the following: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and 14) preventing and/or reversing exhaustion of T cells in a subject in need thereof.

In some embodiments of the use, the cancer or tumor is selected from the group consisting of a hematological cancer, a lymphoma, and a solid tumor. In some embodiments, the cancer or tumor is selected from the group consisting of melanoma, colon cancer, pancreatic cancer, breast cancer, lung cancer, and liver cancer.

In one aspect, the present application provides use of an agent capable of attenuating an expression and/or activity of YTHDF2 in combination with an additional active ingredient in the manufacture of a medicament for one or more of the following: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and 14) preventing and/or reversing exhaustion of T cells in a subject in need thereof.

In some embodiments of the use, the additional active ingredient comprises a cancer immunotherapy. In some embodiments, the additional active ingredient comprises an immune checkpoint inhibitor. In some embodiments, the additional active ingredient comprises an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO inhibitor. In some embodiments, the additional active ingredient comprises pembrolizumab, nivolumab, cemiplimab, atezolizumab, avelumab, durvalumab, and/or ipilimumab.

In one aspect, the present application provides a modified immune cell or a population of cells of the present application, for one or more of the following: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and 14) preventing and/or reversing exhaustion of T cells in a subject in need thereof.

In one aspect, the present application provides a composition of the present application, for one or more of the following: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and 14) preventing and/or reversing exhaustion of T cells in a subject in need thereof.

In one aspect, the present application provides an agent capable of attenuating an expression and/or activity of YTHDF2 of the present application, for one or more of the following: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and 14) preventing and/or reversing exhaustion of T cells in a subject in need thereof.

In one aspect, the present application provides a combination comprising an agent capable of attenuating an expression and/or activity of YTHDF2 of the present application and an additional active ingredient of the present application for one or more of the following: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and 14) preventing and/or reversing exhaustion of T cells in a subject in need thereof.

In one aspect, the present application provides a method for treating a subject of the present application, the method comprises administering to the subject: 1) an immune cell of the present application (such as a modified immune cell of the present application); 2) an agent capable of attenuating an expression and/or activity of YTHDF2 of the present application; 3) an additional active ingredient of the present application; or any combination thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present application are set forth with particularity in the appended claims. A better understanding of the features and advantages of the present application will be obtained by reference to the following detailed description that sets forth illustrative embodiments, in which the principles of the present application are employed, and the accompanying drawings (also “figure” and “FIG.” herein), of which:

FIGS. 1a-1d illustrate the enhanced anti-tumor effects after attenuating YTHDF2 in T cells.

FIGS. 2a-2g illustrate the enhanced functions of T cells after attenuation of YTHDF2.

FIGS. 3a-3e illustrate reversion of T cell exhaustion and enhanced functions of T cells after attenuation of YTHDF2.

FIGS. 4a-4b illustrate the design of CARs.

FIGS. 5a-5d illustrate the expression of CARs after infecting cells with different doses of viruses.

FIGS. 6a-6d illustrate the expression of CARs in various T cells of the present application.

FIGS. 7a-7b illustrate the tumor cell killing effects of various CAR-T cells of the present application.

FIGS. 8A-8B illustrate the design of CARs and the expression of CARs in various T cells of the present application.

FIG. 9 illustrates the tumor cell killing effects of various CAR-T cells of the present application.

FIG. 10 illustrates the tumor cell killing effects of various CAR-T cells of the present application.

### DETAILED DESCRIPTION

While various embodiments of the present application have been shown and described herein, it will be obvious to those skilled in the art that such embodiments are provided by way of example only. Numerous variations, changes, and substitutions may occur to those skilled in the art without departing from the present application. It should be understood that various alternatives to the embodiments of the present application described herein may be employed.

In one aspect, the present application provides a modified immune cell. Comparing to an unmodified corresponding immune cell, the modified immune cell has attenuated expression and/or activity of YTHDF2. In addition, the modified immune cell may have enhanced anti-tumor activity.

In one aspect, the present application provides a composition. The composition may comprise a modified immune cell of the present application. The composition may also comprise a pharmaceutically acceptable excipient.

In one aspect, the present application provides a composition for stimulating T cell-mediated immune response to a cancer cell. In one aspect, the present application provides a composition for stimulating T cell-mediated immune response to a tumor antigen. The composition may comprise an agent capable of attenuating the expression and/or activity of YTHDF2. The composition may further comprise a pharmaceutically acceptable excipient.

In one aspect, the present application provides a composition for treating cancer. The composition may comprise an agent capable of attenuating the expression and/or activity of YTHDF2. The composition may further comprise a pharmaceutically acceptable excipient.

In one aspect, the present application provides a method for activating an immune cell. The method may comprise attenuating an expression and/or activity of YTHDF2 in the immune cell.

In one aspect, the present application provides a method for generating an immune cell having enhanced anti-tumor activity. The method may comprise attenuating an expression and/or activity of YTHDF2 in the immune cells.

In one aspect, the present application provides a method for preventing and/or reversing exhaustion of an immune cell. The method may comprise attenuating an expression and/or activity of YTHDF2 in the immune cells.

In one aspect, the present application provides a method for treating a disease, disorder or condition associated with an expression of a tumor antigen in a subject in need thereof. The method may comprise administering to the subject an agent capable of attenuating an expression and/or activity of YTHDF2.

In one aspect, the present application provides a method for treating a disease, disorder or condition associated with an expression of a tumor antigen in a subject in need thereof. The method may comprise administering to the subject a modified immune cell of the present application.

In one aspect, the present application provides a method for treating a disease, disorder or condition associated with an expression of a tumor antigen in a subject in need thereof. The method may comprise administering to the subject an agent capable of attenuating an expression and/or activity of YTHDF2 and an immune cell (such as a modified immune cell) of the present application.

In one aspect, the present application provides a method for treating cancer in a subject in need thereof. The method may comprise administering to the subject an agent capable of attenuating an expression and/or activity of YTHDF2.

In one aspect, the present application provides a method for treating cancer in a subject in need thereof. The method may comprise administering to the subject a modified immune cell of the present application.

In one aspect, the present application provides a method for treating cancer in a subject in need thereof. The method may comprise administering to the subject an agent capable of attenuating an expression and/or activity of YTHDF2 and an immune cell (such as a modified immune cell) of the present application.

In one aspect, the present application provides a method for stimulating a T cell-mediated immune response to a cancer cell and/or a tumor antigen in a subject in need thereof. The method may comprise administering to the subject an agent capable of attenuating an expression and/or activity of YTHDF2.

In one aspect, the present application provides a method for stimulating a T cell-mediated immune response to a cancer cell and/or a tumor antigen in a subject in need thereof. The method may comprise administering to the subject a modified immune cell of the present application.

In one aspect, the present application provides a method for stimulating a T cell-mediated immune response to a cancer cell and/or a tumor antigen in a subject in need thereof. The method may comprise administering to the subject an agent capable of attenuating an expression and/or activity of YTHDF2 and an immune cell (such as a modified immune cell) of the present application.

In one aspect, the present application provides a method for providing an anti-tumor immunity in a subject in need thereof. The method may comprise administering to the subject an agent capable of attenuating an expression and/or activity of YTHDF2.

In one aspect, the present application provides a method for providing an anti-tumor immunity in a subject in need thereof. The method may comprise administering to the subject a modified immune cell of the present application.

In one aspect, the present application provides a method for providing an anti-tumor immunity in a subject in need thereof. The method may comprise administering to the subject an agent capable of attenuating an expression and/or activity of YTHDF2 and an immune cell (such as a modified immune cell) of the present application.

In one aspect, the present application provides a method for preventing and/or reversing exhaustion of T cells in a subject in need thereof. The method may comprise administering to the subject an agent capable of attenuating an expression and/or activity of YTHDF2.

In one aspect, the present application provides a method for preventing and/or reversing exhaustion of T cells in a subject in need thereof. The method may comprise administering to the subject a modified immune cell of the present application.

In one aspect, the present application provides a method for preventing and/or reversing exhaustion of T cells in a subject in need thereof. The method may comprise administering to the subject an agent capable of attenuating an expression and/or activity of YTHDF2 and an immune cell (such as a modified immune cell) of the present application.

In one aspect, the present application provides use of an agent capable of attenuating an expression and/or activity of YTHDF2 in the manufacture of a composition and/or of a medicament for: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and/or 14) preventing and/or reversing exhaustion of T cells in a subject in need thereof.

In one aspect, the present application provides use of an agent capable of attenuating an expression and/or activity of YTHDF2 in combination with an additional active ingredient in the manufacture of a medicament for: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and/or 14) preventing and/or reversing exhaustion of T cells in a subject in need thereof.

In one aspect, the present application provides a modified immune cell or a population of cells of the present application, for: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and/or 14) preventing and/or reversing exhaustion of T cells in a subject in need thereof.

In one aspect, the present application provides a composition of the present application, for: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and/or 14) preventing and/or reversing exhaustion of T cells in a subject in need thereof.

In one aspect, the present application provides an agent capable of attenuating an expression and/or activity of YTHDF2 of the present application, for: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and/or 14) preventing and/or reversing exhaustion of T cells in a subject in need thereof.

In one aspect, the present application provides a combination comprising an agent capable of attenuating an expression and/or activity of YTHDF2 of the present application and an additional active ingredient of the present application for: 1) activating an immune cell; 2) generating an immune cell having enhanced anti-tumor activity; 3) preventing and/or reversing exhaustion of an immune cell; 4) treating a disease, disorder or condition associated with an expression of a tumor antigen in a subject in need thereof; 5) treating cancer in a subject in need thereof; 6) stimulating a T cell-mediated immune response to a cancer cell and/or a tumor antigen in a subject in need thereof; 7) providing an anti-tumor immunity in a subject in need thereof; 8) increasing and/or improving proliferation of CD4⁺ T cells; 9) increasing and/or improving proliferation of CD8⁺ T cells; 10) increasing the number of CD8⁺ cytotoxic T cells in or surrounding the site of a tumor; 11) increasing the number of tumor infiltrating CD8⁺ T cells; 12) enhancing cytokine production of T cells; 13) enhancing the antitumor response of a cancer immunotherapy; and/or 14) preventing and/or reversing exhaustion of T cells in a subject in need thereof.

In one aspect, the present application provides a method for treating a subject of the present application. The method may comprise administering to the subject: 1) an immune cell of the present application (such as a modified immune cell of the present application); 2) an agent capable of attenuating an expression and/or activity of YTHDF2 of the present application; and/or 3) an additional active ingredient of the present application. For example, the method may comprise administering to the subject 1) an immune cell of the present application (such as a modified immune cell of the present application) and 2) an agent capable of attenuating an expression and/or activity of YTHDF2 of the present application. As another example, the method may comprise administering to the subject 1) an immune cell of the present application (such as a modified immune cell of the present application) and 3) an additional active ingredient of the present application. As another example, the method may comprise administering to the subject 2) an agent capable of attenuating an expression and/or activity of YTHDF2 of the present application and 3) an additional active ingredient of the present application. As another example, the method may comprise administering to the subject 1) an immune cell of the present application (such as a modified immune cell of the present application); 2) an agent capable of attenuating an expression and/or activity of YTHDF2 of the present application; and 3) an additional active ingredient of the present application.

The terms “comprise(s),” “include(s),” “having,” “has,” “can,” “contain(s),” and variants thereof, as used herein, generally are intended to be open-ended transitional phrases, terms, or words that do not preclude the possibility of additional acts or structures. The singular forms “a,” “and” and “the” include plural references.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4. 6.5, 6.6, 6.7, 6.8, 6.9, and 7.0 are explicitly contemplated. Accordingly, the description in range format is merely for convenience and brevity and should not be construed as an inflexible limitation on the scope of the present applications of the present application. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-100 (e.g., 95-96, 95-97, 95-98, 95-99, 95-99.5 or more) % identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate.

The term “subject”, as used herein, generally refers to a human being or an animal. For example, it may refer to any vertebrate, including, but not limited to, a mammal (e.g., cow, pig, camel llama, horse, goat, rabbit, sheep. hamsters, guinea pig, cat, dog. rat, and mouse, a non-human primate (for example, a monkey, such as a cynomolgus monkey, chimpanzee, etc.) and a human). In some aspects, the subject is a human being.

The term “treat”, “treated” and “treating” may be used interchangeably herein, and generally refer to a therapeutic method wherein the object is to slow down (lessen) an undesired physiological condition, disorder or disease, or to obtain beneficial or desired clinical results. In some aspects of the present disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment.

The terms “modified”, “modify” and “modification” may be used interchangeably herein, and generally refer to introducing or resulting in a change or alteration. When used in the context of a gene, such modification may include any conventional method for producing an alteration in the genetic makeup of the target cell or subject. For example, mutagenesis via ultraviolet light irradiation, chemical mutagenesis, targeted mutagenesis such as site directed mutagenesis of a cell (e.g., an immune cell), and creation of a transgenic mouse.

The terms “attenuating”, “attenuation” and “attenuated” may be used interchangeably, and as used herein, may refer to inhibiting or reducing the amount of or inhibiting or decreasing the activity of a target gene or a target protein (such as YTHDF2). Such attenuation may be accomplished using, e.g. an antibody or a derivative thereof, an antibody-drug conjugate, a fusion protein, a small molecule, an antisense molecule, a dsRNA, a siRNA, a shRNA, an aptamer, and/or a gRNA (e.g., in combination with a gene editing system, such as with CRIPSR/Cas9). As another example, YTHDF2 may be attenuated by contacting the immune cell with an inhibitor of YTHDF2, to inhibit binding and/or recognizing of the m6A modified mRNA by YTHDF2.

The term “small molecule”, as used herein, generally refers to any chemical or other moiety, other than polypeptides and nucleic acids, that can act to affect biological processes, particularly to modulate the m6A mRNA modification (e.g., activity of YTHDF2). Small molecules can include any number of therapeutic agents presently known and used, or that can be synthesized in a library of such molecules for the purpose of screening for biological function(s). Small molecules are distinguished from macromolecules by size. The small molecules may have a molecular weight less than about 5,000 daltons (Da), such as less than about 2,500 Da, less than about 1,000 Da, or less than about 500 Da. Small molecules may include without limitation organic compounds, peptidomimetics and conjugates thereof.

The term “organic compound”, as used herein, generally refers to any carbon-based compound other than macromolecules such as nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, monosaccharides, oligosaccharides, polysaccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocyclic compounds, imidazole, and phenols. An organic compound as used herein also includes nitrated organic compounds and halogenated (e.g., chlorinated) organic compounds.

The terms “peptide,” “polypeptide,” and “protein” may be used interchangeably herein, and may generally refer to a linked sequence of amino acids, which may be natural, synthetic, or a modification or combination of natural and synthetic. The term includes antibodies, antibody mimetics, domain antibodies, lipocalins, and targeted proteases. The term also includes vaccines containing a peptide or peptide fragment intended to raise antibodies against the peptide or peptide fragment.

The term “antibody,” as used herein, generally refers to a protein, or polypeptide sequence derived from an immunoglobulin molecule which specifically binds to an antigen. Antibodies can be polyclonal or monoclonal, multiple or single chain, or intact immunoglobulins, and may be derived from natural sources or from recombinant sources. Antibodies can be tetramers of immunoglobulin molecules. Antibodies may be conjugated with a chemical moiety. The antibody may be a human or humanized antibody.

The term “antibody fragment”, as used herein, generally refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005).

The term “antisense” molecule, as used herein, generally refer to antisense or sense oligonucleotides comprising a single-stranded nucleic acid sequence (either RNA or DNA) capable of binding to target mRNA (sense) or DNA (antisense) sequences. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988). Antisense molecules can be modified or unmodified RNA, DNA, or mixed polymer oligonucleotides. These molecules may function by specifically binding to matching sequences resulting in inhibition of peptide synthesis (Wu-Pong, November 1994, BioPharm, 20-33) either by steric blocking or by activating an RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190). In addition, binding of single stranded DNA to RNA can result in nuclease-mediated degradation of the heteroduplex (Wu-Pong, supra). Backbone modified DNA chemistry, which have thus far been shown to act as substrates for RNase H are phosphorothioates, phosphorodithioates, borontrifluoridates, and 2′-arabino and 2′-fluoro arabino-containing oligonucleotides.

The term “siRNA”, as used herein, generally refers to small interfering RNA, which may be small inhibitory RNA duplexes that induce the RNA interference (RNAi) pathway. (Elbashir, S. M. et al. Nature 411:494-498 (2001); Caplen, N. J. et al. Proc. Natl. Acad. Sci. USA 98:9742-9747 (2001); Harborth, J. et al. J Cell Sci. 114:4557-4565 (2001).) These molecules can vary in length (generally 18-30 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region. As used herein, siRNA molecules are not limited to RNA molecules but further encompass chemically modified nucleotides and non-nucleotides. siRNA gene-targeting may be carried out by transient siRNA transfer into cells (achieved by such classic methods as liposome-mediated transfection, electroporation, or microinjection).

The term “gRNA”, as used herein, generally refers to a guide polynucleotide that is capable of forming a complex with a Cas endonuclease (i.e., a guide polynucleotide/Cas endonuclease complex), wherein said guide polynucleotide/Cas endonuclease complex can direct the Cas endonuclease to a DNA target site, enabling the Cas endonuclease to recognize, bind to, and optionally nick or cleave (introduce a single or double strand break) into the DNA target site. A guide polynucleotide/Cas endonuclease complex herein can comprise Cas protein(s) and suitable polynucleotide component(s) of any of the four known CRISPR systems (Horvath and Barrangou, Science 327: 167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occur if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component.

The term “CAR-T cell” as used herein, generally refers to a T cell comprising and/or expressing a chimeric antigen receptor (CAR) molecule. A “CAR molecule”, as used herein, generally refers to a CAR (e.g., a CAR polypeptide), a nucleic acid encoding a CAR, or both.

The term “YTHDF2”, as used herein, generally refers to YTH N6-Methyladenosine RNA Binding Protein 2 or a functional fragment thereof specifically recognizing and binding N6-methyladenosine (m6A)-containing RNAs, and regulating mRNA stability. The human and murine amino acid and nucleic acid sequences can be found in a public database, such as GenBank, UniProt and Swiss-Prot. For example, the amino acid sequences of human YTHDF2 can be found at Accession Nos. NP_001166299.1; NP_001166599.1; and NP_057342.2, and the mRNA sequences encoding them can be found at Accession Nos. NM_001172828.1; NM_001173128.1; and NM_016258.2.

The term “autologous”, as used herein, generally refers to any material derived from the same individual to whom it is later to be re-introduced into the individual.

The term “allogeneic”, as used herein, generally refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The terms “cancer” and “tumor” are used herein interchangeably, and generally refer to a disease characterized by the uncontrolled growth of aberrant cells. Both terms encompass solid and liquid, e.g., diffuse or circulating, tumors. They include premalignant, as well as malignant cancers and tumors.

The phrase “disease, disorder or condition associated with an expression of a tumor antigen” as described herein, generally includes, but is not limited to, a disease associated with expression of a tumor antigen or condition associated with cells expressing a tumor antigen, e.g., proliferative diseases such as a cancer or malignancy or a precancerous condition such as a myelodysplasia, a myelodysplastic syndrome or a preleukemia; or a noncancer related indication associated with cells which express a tumor antigen. In one embodiment, a cancer associated with expression of a tumor antigen as described herein is a hematological cancer. In one embodiment, a cancer associated with expression of a tumor antigen as described herein is a solid cancer. Further diseases associated with expression of a tumor antigen as described herein include, but not limited to, e.g., atypical and/or non-classical cancers, malignancies, precancerous conditions or proliferative diseases associated with expression of a tumor antigen as described herein. Non-cancer related indications associated with expression of a tumor antigen as described herein include, but are not limited to, e.g., autoimmune disease, inflammatory disorders and transplantation. In some embodiments, the tumor antigen-expressing cells express, or at any time expressed, mRNA encoding the tumor antigen. In an embodiment, the tumor antigen-expressing cells produce the tumor antigen protein (e.g., wild-type or mutant), and the tumor antigen protein may be present at normal levels, elevated levels, or reduced levels.

The term “immune effector cell”, as used herein, generally refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes. “Immune effector function or immune effector response,” as used herein, generally refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The terms “activity” and “activating”, as used herein, generally refer to a specialized function of a cell. The activity of a T cell, for example, may be cytolytic activity or helper activity including the secretion of cytokines.

The term “encoding”, as used herein, generally refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The phrase a “nucleotide sequence encoding an amino acid sequence”, as used herein, generally includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “endogenous”, as used herein, generally refers to any material from or produced inside an organism, cell, tissue or system.

The term “exogenous”, as used herein, generally refers to any material introduced from or produced outside an organism, cell, tissue or system.

The term “expression”, as used herein, generally refers to the transcription and/or translation of a particular nucleotide sequence driven by a promoter.

In the context of the present application, the following abbreviations for the commonly occurring nucleic acid bases are used. “A” refers to adenosine, “C” refers to cytosine, “G” refers to guanosine, “T” refers to thymidine, and “U” refers to uridine.

The term “nucleic acid” or “polynucleotide”, as used herein, generally refers to deoxyribonucleic acids (DNA) or ribonucleic acid (RNA), or a combination of a DNA or RNA thereof, and polymers thereof in either single- or double-stranded form. The term “nucleic acid” includes a gene, cDNA or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “cancer associated antigen” and “tumor antigen” are used interchangeably herein and generally refer to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. In some embodiments, a tumor antigen is a marker expressed by both normal cells and cancer cells. In some embodiments, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a cancer-associated antigen is a cell surface molecule that is inappropriately synthesized in the cancer cell, for instance, a molecule that contains deletions, additions or mutations in comparison to the molecule expressed on a normal cell. In some embodiments, a cancer-associated antigen will be expressed exclusively on the cell surface of a cancer cell, entirely or as a fragment (e.g., MHC/peptide), and not synthesized or expressed on the surface of a normal cell.

The term “specifically binds,” as used herein, generally refers to a molecule (e.g., an antibody, or a ligand), which recognizes and binds with a cognate binding partner protein present in a sample, but which molecule does not substantially recognize or bind other molecules in the sample. In some embodiments, a molecule of the present disclosure may specifically bind to a target molecule with a binding affinity (K_{D}) of less than about 10⁻⁶ M (e.g., less than about 5×10⁻⁷ M, less than about 2×10⁻⁷ M, less than about 10⁻⁷ M, less than about 5×10⁻⁸ M, less than about 2×10⁻⁸ M, less than about 10⁻⁸ M, less than about 5×10⁻⁹ M, less than about 4×10⁻⁹M, less than about 3×10⁻⁹M, less than about 2×10⁻⁹ M, or less than about 10⁻⁹ M).

K_{D} may generally refer to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry.

The term “anti-cancer agent”, as used herein, generally refers to an agent that is capable of inhibiting and/or preventing the growth of a tumor or a cancer cell.

The term “CTLA-4”, as used herein, generally refers to the Cytotoxic T-lymphocyte-associated protein 4 derived from any vertebrate source, including mammals such as primates (e.g., humans, monkeys) and rodents (e.g., mice and rats), and functional fragments thereof. Exemplary sequence of human CTLA-4 includes Homo sapiens (human) CTLA-4 protein (NCBI Ref Seq No. AAL07473.1). Exemplary sequence of CTLA-4 includes Macaca fascicularis (monkey) CTLA-4 protein (NCBI Ref Seq No XP_005574071.1). The term “CTLA-4”, as used herein, generally is intended to encompass any form of CTLA-4, for example, 1) native unprocessed CTLA-4 molecule, “full-length” CTLA-4 chain or naturally occurring variants of CTLA-4, including, for example, splice variants or allelic variants; 2) any form of CTLA-4 that results from processing in the cell; or 3) full length, a fragment (e.g., a truncated form, an extracellular/transmembrane domain) or a modified form (e.g. a mutated form, a glycosylated/PEGylated, a His-tag/immunofluorescence fused form) of CTLA-4 subunit generated through recombinant method.

The term “anti-CTLA-4 antibody”, “anti-CTLA-4 binding domain” or “CTLA-4-binding domain” refers to an antibody or antigen-binding domain that is capable of specifically binding CTLA-4 (e.g. human or monkey CTLA-4).

The term “PD-1”, as used herein, generally refers programmed cell death protein, which belongs to the superfamily of immunoglobulin and functions as co-inhibitory receptor to negatively regulate the immune system. PD-1 is a member of the CD28/CTLA-4 family, and has two known ligands including PD-L1 and PD-L2. Representative amino acid sequence of human PD-1 is disclosed under the NCBI accession number: NP_005009.2, and the representative nucleic acid sequence encoding the human PD-1 is shown under the NCBI accession number: NM_005018.2.

The term “PD-L1”, as used herein, generally refers to programmed cell death ligand 1 (PD-L1, see, for example, Freeman et al. (2000) J. Exp. Med. 192: 1027). Representative amino acid sequence of human PD-L1 is disclosed under the NCBI accession number: NP_054862.1, and the representative nucleic acid sequence encoding the human PD-L1 is shown under the NCBI accession number: NM_014143.3. PD-L1 binds to its receptor PD-1 or B7-1, which is expressed on activated T cells, B cells and myeloid cells. The binding of PD-L1 and its receptor induces signal transduction to suppress TCR-mediated activation of cytokine production and T cell proliferation. Accordingly, PD-L1 plays a major role in suppressing immune system during particular events such as pregnancy, autoimmune diseases, tissue allografts, and is believed to allow tumor or cancer cells to circumvent the immunological checkpoint and evade the immune response.

The term “anti-PD-1 antibody”, “anti-PD-1 binding domain” or “PD-1 binding domain” as used herein, generally refers to an antibody or antigen-binding domain that is capable of specifically binding to PD-1 (e.g. human or monkey PD-1) with an affinity which is sufficient to provide for diagnostic and/or therapeutic use.

The term “anti-tumor immunity”, as used herein, generally refers to an immune response induced upon recognition of cancer antigens by immune cells.

The term “cancer immunotherapy”, as used herein, generally refers to any therapy that is designed to provoke or enhance an immune response against cancer cells in a patient. For example, cancer immunotherapy includes, but is not limited to, cancer antigen specific active immunotherapy, treatment with an immunomodulator (e.g., an activator or an inhibitor of an immune suppressor or an inhibitor of a checkpoint inhibitor), or treatment with a cancer cell or a mixture of antigens derived therefrom (e.g., treatment with antigens derived from a cancer cell line). Cancer immunotherapy includes a therapeutic treatment that stimulates or restores the ability of the immune system to fight cancer by inducing, enhancing or suppressing an immune response. Cancer immunotherapy results in targeting of an immune activity against a disease-specific antigen, either by increasing immune cell recognition of the target or by reducing disease-related immune suppression.

The term “tumor infiltrating T cell”, as used herein, generally refers to a T cell that infiltrates tumors. The tumor infiltrating T cells may appear naturally reactive to autologous tumor antigens. These cells can be found in the tumor stroma and/or within the tumor itself.

The term “TCR-T cell”, as used herein, generally refers to a T cell comprising or expressing an engineered and/or modified T cell receptor.

The term “IDO inhibitor”, as used herein, generally refers to an agent capable of inhibiting the activity of indoleamine 2,3-dioxygenase (IDO) and thereby reversing IDO-mediated immunosuppression. The IDO inhibitor may inhibit IDO1 and/or IDO2 (INDOL1). An IDO inhibitor may be a reversible or irreversible IDO inhibitor. “A reversible IDO inhibitor” is a compound that reversibly inhibits IDO enzyme activity either at the catalytic site or at a non-catalytic site and “an irreversible IDO inhibitor” is a compound that irreversibly destroys IDO enzyme activity by forming a covalent bond with the enzyme.

The term “immune checkpoint inhibitor”, as used herein, generally refers to any molecule that directly or indirectly inhibits, partially or completely, an immune checkpoint pathway. It is generally thought that immune checkpoint pathways function to turn on or off aspects of the immune system, particularly T cells, but also for instance myeloid cells, NK cells and B cells. Following activation of a T cell, a number of inhibitory receptors can be upregulated and present on the surface of the T cell in order to suppress the immune response at the appropriate time. Examples of immune checkpoint pathways include, without limitation, PD-1/PD-L1, CTLA-4/B7-1, TIM-3, LAG3, B7-H1, H4, HAVCR2, IDO1, CD276 and VTCN1, B7-H3, B7-H4, CD47, and MR. For instance, non-limiting examples of immune checkpoint inhibitors or modulators include fully human monoclonal antibodies, such as BMS-936558/MDX-1106, BMS-s936559/MDX-1105, ipilimumab/Yervoy, tremelimumab, BMS-986016, Durvalumab, MEDI4736, Urelumab, CDX-1127, and Avelumab; humanized antibodies, such as CT-011, IV1K-3475, Hu5F9-G4, CC-90002, MBG453, TSR-022, and Atezolizumab; and fusion proteins, such as AMP-224 and TTI-621, and others. Other non-limiting examples of immune checkpoint modulators (agonists) include antibodies directed against e.g., CD40, OX40, GITR, CD137 (4-1 BB), CD27, ICOS, and TRAIL. In accordance with this invention, the one or more immune checkpoint modulator(s) may independently be a polypeptide or a polypeptide-encoding nucleic acid molecule; said polypeptide comprising a domain capable of binding the targeted immune checkpoint and/or inhibiting the binding of a ligand to said targeted immune checkpoint so as to exert an antagonist function (i.e. being capable of antagonizing an immune checkpoint-mediated inhibitory signal) or an agonist function (i.e. being capable of boosting an immune checkpoint-mediated stimulatory signal). Such one or more immune checkpoint modulator(s) can be independently selected from the group consisting of peptides (e.g., peptide ligands), soluble domains of natural receptors, RNAi, antisense molecules, antibodies and protein scaffolds. For example, the immune checkpoint modulator may be an antibody. In the context of the present disclosure, the immune check modulator antibody is used in the broadest sense and encompasses e.g., naturally occurring and engineered by man as well as full length antibodies or functional fragments or analogs thereof that are capable of binding the target immune checkpoint or epitope (thus retaining the target-binding portion). It can be of any origin, e.g., human, humanized, animal (e.g. rodent or camelid antibody) or chimeric. It may be of any isotype with a specific preference for an IgG1 or IgG4 isotype. In addition, it may be glycosylated or non-glycosylated. Standard assays to evaluate the binding ability of the antibodies toward immune checkpoints are known in the art, including for example, ELISAs, Western blots, RIAs and flow cytometry. The binding kinetics (e.g., binding affinity) of the antibodies also can be assessed by standard assays known in the art, such as by Biacore analysis. Where in the application reference is made to an immune checkpoint inhibitor, also an immune checkpoint modulator may be used, except in those cases where it is apparent from the context of the wording that this is not the case.

The term “exhaustion”, as used herein, generally refers to T cell exhaustion, which is a state of T-cell dysfunction that arises during many chronic infections and cancer. T cell exhaustion is characterized by poor T-cell effector function, sustained expression of inhibitory receptors and/or a transcriptional state distinct from that of functional effector or memory T-cells. Exhaustion prevents optimal control of infection and tumors. T-cell exhaustion may show a stepwise and progressive loss of T-cell functions. “Reversing exhaustion”, as used herein, generally refers to an activity or capability to restore at least some of the weakened or reduced anti-tumor activity of an exhausted T cell. Reversing exhaustion may also include preventing a T cell from being exhausted.

The term “PROTAC”, as used herein, generally refers to a proteolysis targeting chimera. A PROTAC may be a bifunctional small molecule composed of at least two active domains and is capable of removing specific proteins. A PROTAC may function by inducing intracellular proteolysis of a target protein. For example, a PROTAC may comprise two covalently linked protein-binding molecules: one capable of engaging an E3 ubiquitin ligase, and another is capable of binding to a target protein (e.g., YTHDF2) for degradation. Recruitment of the E3 ligase to the target protein (e.g., YTHDF2) may result in ubiquitination and subsequent degradation of the target protein by the proteasome.

The term “T cell-mediated immune response”, as used herein, generally refers to an immune response influenced by modulation of T cell costimulation. Exemplary immune responses include T cell responses, e.g., cytokine production, and cellular cytotoxicity. In addition, T cell-mediated immune response also includes immune responses that are indirectly affected by T cell activation, e.g., antibody production (humoral responses) and activation of cytokine responsive cells, e.g., macrophages.

The term “Chimeric Antigen Receptor” or “CAR”, as used herein, generally refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the costimulatory molecule is chosen from the costimulatory molecules described herein, e.g., 4-1BB (i.e., CD137) and/or CD28. In one aspect, the CAR comprises a chimeric fusion protein comprising an extracellular antigen binding domain, a transmembrane domain and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one aspect, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one aspect, the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

A CAR that comprises an antigen binding domain (e.g., a scFv, or TCR) that targets a specific tumor maker X, such as those described herein, is also referred to as X CAR. For example, a CAR that comprises an antigen binding domain that targets CD20 is referred to as CD20 CAR or 20 CAR. For example, a CAR that comprises an antigen binding domain that targets CLDN18.2 is referred to as CLDN18.2 CAR.

The term “signaling domain”, as used herein, generally refers to the functional portion of a protein which acts by transmitting information within the cell to regulate cellular activity via defined signaling pathways by generating second messengers or functioning as effectors by responding to such messengers.

The term “scFv”, as used herein, generally refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “binding domain”, as used herein, generally refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., a bispecific antibody molecule.

The portion of the CAR of the present application comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody. In one aspect, the antigen binding domain of a CAR of the present application comprises an antibody fragment. In a further aspect, the CAR comprises an antibody fragment that comprises a scFv.

The term “antigen” or “Ag”, as used herein, generally refers to a molecule that provokes an immune response. This immune response may involve either antibody production, or the activation of specific immunologically-competent cells, or both. The person of ordinary skills in the art will understand that any macromolecule, including virtually all proteins or peptides, can serve as an antigen. Furthermore, antigens can be derived from recombinant or genomic DNA. A person of ordinary skills in the art will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. An antigen need not be encoded by a “gene”. It can be synthesized or can be derived from a biological sample, or might be macromolecule besides a polypeptide. Such a biological sample can include, but is not limited to a tissue sample, a tumor sample, a cell or a fluid with other biological components.

The term “anti-cancer” or “anti-tumor”, as used herein, generally refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer” or “anti-tumor” effect can also be manifested by the ability to prevent of the occurrence of cancer in the first place.

The term “derived from”, as used herein, generally refers to a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3 zeta sequence and delete unwanted sequence, or impose mutations, to arrive at the intracellular signaling domain.

The term “intracellular signaling domain”, as used herein, generally refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

The intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain comprises a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or FTAM. Examples of ITAM containing primary cytoplasmic signaling sequences include, but are not limited to, those derived from CD3 zeta.

The term “zeta” or alternatively “zeta chain”, “CD3-zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO: 8.

The term a “costimulatory molecule”, as used herein, generally refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to CD28, 4-1BB (CD137) and the like. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD28, and 4-1BB (CD137), or the like.

The intracellular signaling domain can comprise the entire intracellular portion, or the entire native intracellular signaling domain, of the molecule from which it is derived, or a functional fragment or derivative thereof.

The term “4-IBB”, as used herein, generally refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like. In one aspect, the “4-1BB costimulatory domain” is the sequence provided as SEQ ID NO: 7 or the equivalent residues from a non-human species, e.g., mouse, rodent, monkey, ape and the like.

The term “Immune effector function” or “immune effector response”, as used herein, generally refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “effective amount” or “therapeutically effective amount”, as used herein, generally refers to an amount of a compound, formulation, material, or composition, as described herein effective to achieve a particular biological result.

The term “expression”, as used herein, generally refers to the transcription and/or translation of a particular nucleotide sequence driven by a promoter.

The term “homologous” or “identity”, as used herein, generally refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “cancer associated antigen” or “tumor antigen”, as used herein, generally refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell.

The term, a “substantially purified” cell, as used herein, generally refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “gene editing system”, as used herein, generally refers to a system, e.g., one or more molecules, that direct and effect an alteration, e.g., a deletion, of one or more nucleic acids at or near a site of genomic DNA targeted by said system. Gene editing systems are known in the art.

The term “dominant negative”, as used herein, generally refers to a gene product or protein that interferes with the function of another gene product or protein. The other gene product affected can be the same or different from the dominant negative protein. Dominant negative gene products can be of many forms, including truncations, full length proteins with point mutations or fragments thereof, or fusions of full length wild type or mutant proteins or fragments thereof with other proteins. The level of inhibition observed can be very low. For example, it may require a large excess of the dominant negative protein compared to the functional protein or proteins involved in a process in order to see an effect. It may be difficult to see effects under normal biological assay conditions. In one embodiment, a dominant negative YTHDF2 cannot bind, recognize, and/or modify m⁶A RNA.

Immune Cells

The immune cell of the present application may be an immune effector cell. For example, the immune cell may be a lymphocyte, such as a T cell. In some cases, the immune cell may be a CD4⁺ cell. In some cases, the immune cell may be a CD8⁺ cell. In some cases, the immune cell may express or comprise tumor specific receptors, such as a tumor specific chimeric antigen receptor, and/or a tumor specific T cell receptor. In some cases, the immune cell may be a tumor infiltrating lymphocyte (e.g., tumor infiltrating T cell). In some cases, the immune cell may be a lymphocyte (e.g., a T cell) obtained from a subject (such as a cancer patient), in some cases, the immune cell may be isolated from a tumor tissue.

The immune cell may be a cell (e.g., a population of cells, such as a population of immune effector cells) engineered to express a chimeric antigen receptor (CAR). In some cases, the immune cell may be a CAR-T cell. In some cases, the immune cell may be a cell (e.g., a population of cells, such as a population of immune effector cells) engineered to express a T cell receptor (such as a tumor specific T cell receptor). In some cases, the immune cell may be a TCR-T cell.

The unmodified immune cell may be TCF1⁻. In some cases, the unmodified immune cell may be Tim3⁺. In some cases, the unmodified immune cell may be PD-1⁺. In some cases, the unmodified immune cell may be TCF1⁻ Tim3⁺. In some cases, the unmodified immune cell may be TCF1⁻ PD-1⁺. In some cases, the unmodified immune cell may be Tim3⁺ PD-1⁺. In some cases, the unmodified immune cell may be TCF1⁻PD-1⁺Tim3⁺.

In some cases, before modification, the percentage of TCF1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁺ cells.

In some cases, before modification, the percentage of Tim3⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁻ cells.

In some cases, before modification, the percentage of PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of PD-1⁻ cells.

In some cases, before modification, the percentage of TCF1⁻ Tim3⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁺Tim3⁻ cells.

In some cases, before modification, the percentage of TCF1⁻PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁺PD-1⁻ cells.

In some cases, before modification, the percentage of Tim3⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁻PD-1⁻ cells.

In some cases, before modification, the percentage of TCF1⁻PD-1⁺Tim3⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁺PD-1⁻ Tim3⁻ cells.

After modification, the modified immune cell may be PD-1⁺ or PD-1⁻. In some cases, the modified immune cell may be TCF1⁺ and/or TCF7⁺. In some cases, the modified immune cell may be Tim3⁻. In some cases, the modified immune cell may be TCF1⁺ Tim3⁻. In some cases, the modified immune cell may be TCF7⁺ Tim3⁻. In some cases, the modified immune cell may be PD-1⁺ Tim3⁻. In some cases, the modified immune cell may be PD-1⁻Tim3⁻. In some cases, the modified immune cell may be Tim3⁻TCF7⁺ TCF1⁺. In some cases, the modified immune cell may be Tim3⁻TCF1⁺PD-1⁺. In some cases, the modified immune cell may be Tim3⁻TCF1⁺PD-1⁺. In some cases, the modified immune cell may be Tim3⁻TCF7⁺PD-1⁺. In some cases, the modified immune cell may be Tim3⁻TCF7⁺PD-1⁻. In some cases, the modified immune cell may be Tim3⁻TCF7⁺ TCF1⁺PD-1⁺. In some cases, the modified immune cell may be Tim3⁻ TCF7⁺ TCF1⁺PD-1⁻. In some cases, the modified immune cell may be TCF7⁺ TCF1⁺. In some cases, the modified immune cell may be TCF7⁺PD-1⁺. In some cases, the modified immune cell may be TCF7⁺PD-1⁻. In some cases, the modified immune cell may be TCF7⁺ TCF1⁺PD-1⁻. In some cases, the modified immune cell may be TCF7⁺ TCF1⁺PD-1⁺. In some cases, the modified immune cell may be TCF1⁺PD-1⁻. In some cases, the modified immune cell may be TCF1⁺PD-1⁺.

In some cases, after modification, the percentage of TCF1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁻ cells.

In some cases, after modification, the percentage of TCF7⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁻ cells.

In some cases, after modification, the percentage of PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of PD-1⁻ cells.

In some cases, after modification, the percentage of PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of PD-1⁺ cells.

In some cases, after modification, the percentage of Tim3⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁺ cells.

In some cases, after modification, the percentage of TCF1⁺Tim3⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁻Tim3⁺ cells.

In some cases, after modification, the percentage of TCF7⁺ Tim3⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁻Tim3⁺ cells.

In some cases, after modification, the percentage of PD-1⁺ Tim3⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of PD-1⁻Tim3⁺ cells.

In some cases, after modification, the percentage of PD-1⁻Tim3⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of PD-1⁺Tim3⁺ cells.

In some cases, after modification, the percentage of Tim3⁻ TCF7⁺TCF1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁺TCF7⁻TCF1⁻ cells.

In some cases, after modification, the percentage of Tim3⁻TCF1⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁺TCF1⁻PD-1⁻ cells.

In some cases, after modification, the percentage of Tim3⁻TCF1⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁺TCF1⁻PD-1⁺ cells.

In some cases, after modification, the percentage of Tim3⁻TCF7⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁺TCF7⁻PD-1⁻ cells.

In some cases, after modification, the percentage of Tim3⁻TCF7⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁺TCF7⁻PD-1⁺ cells.

In some cases, after modification, the percentage of Tim3⁻TCF7⁺TCF1⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁺TCF7⁻TCF1⁻PD-1⁻ cells.

In some cases, after modification, the percentage of Tim3⁻TCF7⁺TCF1⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁺TCF7⁻TCF1⁻PD-1⁺ cells.

In some cases, after modification, the percentage of TCF7⁺TCF1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁻TCF1⁻ cells.

In some cases, after modification, the percentage of TCF7⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁻PD-1⁻ cells.

In some cases, after modification, the percentage of TCF7⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁻PD-1⁺ cells.

In some cases, after modification, the percentage of TCF7⁺TCF1⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁻TCF1⁻PD-1⁺ cells.

In some cases, after modification, the percentage of TCF7⁺TCF1⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁻TCF1⁻PD-1⁻ cells.

In some cases, after modification, the percentage of TCF1⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁻PD-1⁺ cells.

In some cases, after modification, the percentage of TCF1⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁻PD-1⁻ cells.

In some cases, after modification, the percentage of TCF1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁺ cells in the corresponding population of immune cells prior to said modification.

In some cases, after modification, the percentage of TCF7⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁺ cells in the corresponding population of immune cells prior to said modification.

In some cases, after modification, the percentage of PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of PD-1⁺ cells in the corresponding population of immune cells prior to said modification.

In some cases, after modification, the percentage of PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of PD-1⁻ cells in the corresponding population of immune cells prior to said modification.

In some cases, after modification, the percentage of Tim3⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁻ cells in the corresponding population of immune cells prior to said modification.

In some cases, after modification, the percentage of TCF1⁺ Tim3⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁺ Tim3⁻ cells in the corresponding population of immune cells prior to said modification.

In some cases, after modification, the percentage of TCF7⁺ Tim3⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁺ Tim3⁻ cells in the corresponding population of immune cells prior to said modification.

In some cases, after modification, the percentage of PD-1⁺Tim3⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of PD-1⁺ Tim3⁻ cells in the corresponding population of immune cells prior to said modification.

In some cases, after modification, the percentage of PD-1⁻Tim3⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of PD-1⁻Tim3⁻ cells in the corresponding population of immune cells prior to said modification.

In some cases, after modification, the percentage of Tim3⁻TCF7⁺ TCF1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁻TCF7⁺TCF1⁺ cells in the corresponding population of immune cells prior to said modification.

In some cases, after modification, the percentage of Tim3⁻TCF1⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁻TCF1⁺PD-1⁺ cells in the corresponding population of immune cells prior to said modification.

In some cases, after modification, the percentage of Tim3⁻TCF1⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁻TCF1⁺PD-1⁻ cells in the corresponding population of immune cells prior to said modification.

In some cases, after modification, the percentage of Tim3⁻TCF7⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁻TCF7⁺PD-1⁺ cells in the corresponding population of immune cells prior to said modification.

In some cases, after modification, the percentage of Tim3⁻TCF7⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁻TCF7⁺PD-1⁻ cells in the corresponding population of immune cells prior to said modification.

In some cases, after modification, the percentage of Tim3⁻TCF7⁺TCF1⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁻TCF7⁺TCF1⁺PD-1⁺ cells in the corresponding population of immune cells prior to said modification.

In some cases, after modification, the percentage of Tim3⁻TCF7⁺TCF1⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of Tim3⁻TCF7⁺TCF1⁺PD-1⁻ cells in the corresponding population of immune cells prior to said modification.

In some cases, after modification, the percentage of TCF7⁺TCF1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁺ TCF1⁺ cells in the corresponding population of immune cells prior to said modification.

In some cases, after modification, the percentage of TCF7⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁺PD-1⁺ cells in the corresponding population of immune cells prior to said modification.

In some cases, after modification, the percentage of TCF7⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁺PD-1⁻ cells in the corresponding population of immune cells prior to said modification.

In some cases, after modification, the percentage of TCF7⁺TCF1⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁺ TCF1⁺PD-1⁻ cells in the corresponding population of immune cells prior to said modification.

In some cases, after modification, the percentage of TCF7⁺TCF1⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF7⁺TCF1⁺PD-1⁺ cells in the corresponding population of immune cells prior to said modification.

In some cases, after modification, the percentage of TCF1⁺PD-1⁻ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁺PD-1⁻ cells in the corresponding population of immune cells prior to said modification.

In some cases, after modification, the percentage of TCF1⁺PD-1⁺ cells in a population of immune cells may be higher (e.g., at least about 1% higher, at least about 2% higher, at least about 3% higher, at least about 4% higher, at least about 5% higher, at least about 8% higher, at least about 10% higher at least about 15% higher, at least about 16% higher, at least about 17% higher, at least about 18% higher, at least about 19% higher, at least about 20% higher, at least about 25% higher, at least about 30% higher, at least about 35% higher, at least about 40% higher, at least about 45% higher, at least about 50% higher, at least 60% higher, at least 70% higher, at least 80% higher, at least 90% higher, at least 100% higher, at least 1.5 folds higher, at least 2 folds higher, at least 2.5 folds higher, at least 3 folds higher, or more) than that of TCF1⁺PD-1⁺ cells in the corresponding population of immune cells prior to said modification.

The immune cell may have been modified with an agent capable of attenuating the expression and/or activity of YTHDF2. For example, in a population of immune cells, one or more cells may have been modified with an agent capable of attenuating the expression and/or activity of YTHDF2. In some embodiments, the immune cells (e.g., engineered or modified immune effector cells, e.g., T cells) of the present application may comprise an agent capable of attenuating the expression and/or activity of YTHDF2.

The agent capable of attenuating the expression and/or activity of YTHDF2 may be introduced into the immune cells, (conditionally) activated in the immune cells, and/or induced to be expressed in the immune cells. In some cases, the agent capable of attenuating the expression and/or activity of YTHDF2 may be allowed to be in contact with the immune cells for a period of time sufficient to result in an reduced expression and/or activity of YTHDF2, for example, the agent may be administered in a medium for culturing the immune cells.

The immune cell may have been subjected to a modification that results in a complete or partial deletion, a complete or partial replacement and/or reduced expression of a gene expressing YTHDF2. For example, in a population of immune cells, one or more cells have been subjected to a modification that results in a complete or partial deletion, a complete or partial replacement and/or reduced expression of a gene expressing YTHDF2. For example, such a modification may comprise homologous recombination, through the use of a nucleic acid molecule (e.g., vector) containing at least a portion of a Ythdf2 gene into which a deletion, addition or substitution has been introduced to thereby alter, e.g., functionally disrupt, the Ythdf2 gene. The Ythdf2 gene can be a human gene, or a nonhuman homologue of a human Ythdf2 gene. For example, a mouse Ythdf2 gene can be used to construct a homologous recombination vector suitable for altering an endogenous Ythdf2 gene, respectively, in the mouse genome. In some embodiments, the vector may be designed such that, upon homologous recombination, the endogenous Ythdf2 gene is functionally disrupted (i.e., no longer encodes a functional protein; also referred to as a “knock out” vector). Alternatively, the vector can be designed such that, upon homologous recombination, the endogenous Ythdf2 gene is mutated or otherwise altered but still encodes functional protein (e.g., the upstream regulatory region can be altered to thereby alter the expression of the endogenous Ythdf2 protein). In the homologous recombination vector, the altered portion of the Ythdf2 gene may be flanked at its 5′ and 3′ ends by additional nucleic acid of the Ythdf2 gene to allow for homologous recombination to occur between the exogenous Ythdf2 gene carried by the vector and an endogenous Ythdf2 gene a cell (e.g., an immune cell). The additional flanking Ythdf2 nucleic acid may be of sufficient length for successful homologous recombination with the endogenous gene. Typically, several kilobases of flanking DNA (both at the 5′ and 3′ ends) may be included in the vector. The vector may be introduced into the immune cells (e.g., by electroporation) and cells in which the introduced Ythdf2 gene has homologously recombined with the endogenous Ythdf2 gene may be selected.

In some cases, the modification is not applied directly to the immune cell (e.g., immune effector cell, such as T cell) itself, instead, the immune cell may be derived from (e.g., differentiated from, as a progeny of, etc.) a cell (e.g., a progenitor of an immune cell) or an organism that has been subjected to modifications resulting in a complete or partial deletion, a complete or partial replacement and/or reduced expression of a gene expressing YTHDF2 in said cell or said organism.

Such modified cell (e.g., immune cell or its progenitors) or organism (e.g., a transgenic nonhuman animal) may contain selected systems which allow for regulated expression and/or regulated deletion of a gene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355.

In some cases, the immune cells (e.g., engineered or modified immune effector cells, e.g., T cells) may comprise a nucleic acid molecule encoding a CAR or a TCR. For example, the nucleic acid molecule may be directly transduced into the immune cell. In some cases, the nucleic acid molecule may be introduced into the immune cells via a carrier (e.g., liposome or a viral vector). The nucleic acid molecule may be capable of expressing the CAR or the TCR in mammalian immune effector cells (e.g., T cells).

The immune cells may be human cells, such as human T cells.

In some cases, prior to expansion, genetic modification or other modification, a source of the cells, e.g., the immune cell (such as the T cell) or a progenitor cell thereof may be obtained from a subject. The term “subject” herein is intended to include living organisms in which an immune response can be elicited (e.g., mammals). Examples of subjects include humans, monkeys, chimpanzees, dogs, cats, mice, rats, and transgenic species thereof. The immune cells or progenitors thereof may be obtained from a number of sources, including peripheral blood mononuclear cells, bone marrow, lymph node tissue, cord blood, thymus tissue, tissue from a site of infection, ascites, pleural effusion, spleen tissue, and/or tumors.

Chimeric Antigen Receptor (CAR)

The immune cells of the present application may comprise and/or express a CAR, and/or a nucleic acid molecule encoding for a CAR. The CAR may comprise an antigen binding domain (e.g., antibody or antibody fragment, TCR or TCR fragment) that binds specifically to a cancer associated antigen described herein, wherein the sequence of the antigen binding domain may be contiguous with and in the same open reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule.

For example, a CAR used in the present application may comprise an antigen-binding domain, a transmembrane domain, and an intracellular signaling domain. The antigen-binding domain may bind to a tumor antigen (e.g., CD20 or CLDN18.2). As an example, the antigen-binding domain may comprise or be an antibody or antibody fragment derived from Rituximab.

The transmembrane domain of the CAR may comprise: (i) an amino acid sequence having at least one, two or three modifications but not more than 20, 10 or 5 modifications of an amino acid sequence of SEQ ID NO: 10, or a sequence with 95-100 (e.g., 95-96, 95-97, 95-98, 95-99, 95-99.5 or more) % identity to an amino acid sequence of SEQ ID NO: 10; or (ii) the sequence of SEQ ID NO: 10.

The antigen binding domain of the CAR may be connected to the transmembrane domain by a hinge region. The hinge region may comprise SEQ ID NO: 9, or a sequence with 95-100 (e.g., 95-96, 95-97, 95-98, 95-99, 95-99.5 or more) % identity thereof.

The intracellular signaling domain of the CAR may comprise a primary signaling domain and/or a costimulatory signaling domain. The primary signaling domain may comprise a functional signaling domain of CD3 zeta. In some cases, the primary signaling domain of the CAR may comprise: (i) an amino acid sequence having at least one, two or three modifications but not more than 20, 10 or 5 modifications of an amino acid sequence of SEQ ID NO: 8, or a sequence with 95-100 (e.g., 95-96, 95-97, 95-98, 95-99, 95-99.5 or more) % identity to an amino acid sequence of SEQ ID NO: 8; or (ii) the amino acid sequence of SEQ ID NO: 8.

The intracellular signaling domain of the CAR may comprise a costimulatory signaling domain, or a primary signaling domain and a costimulatory signaling domain. The costimulatory signaling domain may comprise a functional signaling domain of 4-1BB (CD137). In some cases, the costimulatory signaling domain of the CAR may comprise an amino acid sequence having at least one, two or three modifications but not more than 20, 10 or 5 modifications of an amino acid sequence of SEQ ID NO: 7, or a sequence with 95-100 (e.g., 95-96, 95-97, 95-98, 95-99, 95-99.5 or more) % identity to an amino acid sequence of SEQ ID NO: 7.

In some cases, the intracellular domain of the CAR may comprise the sequence of SEQ ID NO: 7, and the sequence of SEQ ID NO: 8, wherein the sequences comprising the intracellular signaling domain are expressed in the same open reading frame and as a single polypeptide chain.

As an example, a CAR of the present application may comprise a scFv domain. The scFv may be followed by an optional hinge sequence such as provided in SEQ ID NO: 9, a transmembrane region such as provided in SEQ ID NO: 10, an intracellular signaling domain such as provided in SEQ ID NO: 7 and a CD3 zeta sequence that includes e.g. SEQ ID NO: 8, e.g., wherein the domains may be contiguous with each other and in the same open reading frame to form a single fusion protein.

An exemplary CAR construct may comprise an optional leader sequence, an extracellular antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein).

Another exemplary CAR construct may comprise an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

An exemplary hinge/spacer sequence is provided as SEQ ID NO: 9. An exemplary transmembrane domain sequence is provided as SEQ ID NO: 10. An exemplary sequence of the intracellular signaling domain of the 4-IBB protein is provided as SEQ ID NO: 7. An exemplary CD3zeta domain sequence is provided as SEQ ID NO: 8.

Antigen Binding Domain

The CAR of the present application may comprise a target-specific binding element, which may also be referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR of the present application include those associated with viral, bacterial and parasitic infections, autoimmune disease and cancer cells.

For example, CAR-mediated T-cell response can be directed to an antigen of interest by way of engineering an antigen binding domain that specifically binds a desired antigen into the CAR.

For example, the portion of the CAR comprising the antigen binding domain may comprise an antigen binding domain that targets a tumor antigen, e.g., a tumor antigen described herein.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

As an example, the 20 CAR is a CD20 CAR comprising an antigen binding domain specifically binding to CD20. In some cases, the antigen binding domain against CD20 is or comprises an antigen binding portion, e.g., CDRs, of the antibody Rituximab, Ofatumumab, Ocrelizumab, Veltuzumab, or GA101.

The antigen binding domain may comprise one, two, three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above.

In some cases, the antigen binding domain may comprise a heavy chain variable region and/or a variable light chain region of an antibody listed above.

In some cases, the antigen binding domain may comprise a humanized antibody or an antibody fragment.

In some cases, a non-human antibody may be humanized, where specific sequences or regions of the antibody may be modified to increase similarity to an antibody naturally produced in a human or fragment thereof. For example, the antigen binding domain may be humanized.

The antigen binding domain of the CAR may specifically bind a tumor antigen as described herein.

The antigen binding domain of the CAR may comprise an scFv, and the scFv may comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. For example, the linker sequence may comprise the amino acids glycine and serine. As another example, the linker sequence may comprise sets of glycine and serine repeats such as (Gly₄Ser)ₙ, where n is a positive integer equal to or greater than 1. For example, n may be 1, 2, 3, 4, 5, 6, 7, 8, 9, or more.

In some cases, the antigen binding domain may be a T cell receptor (TCR), or a fragment thereof, for example, a single chain TCR (scTCR). For example, scTCR can be engineered to contain the vα and vβ genes from a T cell clone linked by a linker (e.g., a flexible peptide).

Sometimes, the proteins of the present application may be produced, e.g., by the use of separate promoters, or by the use of a bicistronic transcription product (which can result in the production of two proteins by cleavage of a single translation product or by the translation of two separate protein products). For example, a CAR of the present application and a YTHDF2 attenuating agent (e.g., a dominant negative YTHDF2 protein) may be produced as a bicistronic transcription product. For example, a sequence encoding a cleavable peptide, e.g., a P2A or F2A sequence, may be disposed between a first protein and a second protein. Examples of peptide cleavage sites include the following, wherein the GSG residues are optional: T2A (SEQ ID NO: 19), P2A (SEQ ID NO: 16), E2A (SEQ ID NO: 20), and/or F2A (SEQ ID NO: 21).

Attenuating the Expression and/or Activity of YTHDF2

The present application provides various approaches to attenuate the expression and/or activity of YTHDF2.

For example, an agent capable of attenuating the expression and/or activity of YTHDF2 may be employed in the present application. It may comprise an agent capable of attenuating the expression of a gene encoding YTHDF2. It may comprise an agent capable of attenuating an activity of a gene encoding YTHDF2. It may comprise an agent capable of attenuating the expression of the YTHDF2 protein. It may comprise an agent capable of attenuating the activity of the YTHDF2 protein.

In some cases, the YTHDF2 attenuating agent may also attenuate the expression and/or activity of a target other than YTHDF2. In some cases, the YTHDF2 attenuating agent may enhance or increase the activity and/or expression of a target other than YTHDF2.

Such an attenuating agent may be a macromolecule. A macromolecule may be a naturally occurring or chemically synthesized organic or inorganic molecule that is greater than or equal to about a 1000 Daltons to about or greater than 1, 2, 3, 5, 7, 10 or more trillion Daltons. A macromolecule may contain two or more monomeric subunits, or derivatives thereof, which are linked by a covalent bond, an ionic bond, or other chemical interactions, such as hydrogen bonding, ionic pairing, base pairing or pairing between charges formed by charge polarization. The monomeric subunits can be different from one another, or identical to one another, and, in some embodiments, can form a polymer. A macromolecule may also be a molecule that, regardless of whether it has more than one subunit and/or is a polymer, can form tertiary and/or quaternary structure. Examples of macromolecules include a polynucleotide, a nucleic acid molecule including DNA, RNA, including siRNA, snRNA, tRNA, antisense RNA, and ribozymes, peptide nucleic acid (PNA), a polypeptide, glycopeptides, a protein, a carbohydrate, or a lipid, or derivatives or combinations thereof, for example, a nucleic acid molecule containing a peptide nucleic acid portion or a glycoprotein, respectively. Examples of macromolecules further include macromolecular assemblies, for examples, viruses, virus particles, phages, viroids, prions and combinations and conjugates thereof.

Such an attenuating agent may be a small molecule. A small molecule may be a naturally occurring or chemically synthesized organic or inorganic molecule that is less than about 1000 Daltons, from about or at 1000 Daltons to about or at 950, 900, 850, 800, 750, 700, 650, 600, 550, 500, 450, 400, 375, 350, 325, 300, 275, 250, 225, 200, 175, 150, 125, 100, 75, 70, 65, 60, 55, 50, 45, 40, 35, 30, 25, 20, 15, 10, 5 or less Daltons. A small molecule may be any molecule that is not a macromolecule, such as a protein or nucleic acid. A “small molecule” can include a molecule containing two or more monomeric subunits, such as a dipeptide or dinucleotide. For example, the YTHDF2 inhibitor may inhibit the N6-methyladenosine (m6A) binding domain of the YTH domain-containing family protein. For example, the YTHDF2 inhibitor may inhibit the YTHDF2 and m6A interaction with an IC₅₀ of 10000 nM or less, 1000 nM or less, 100 nM or less, 10 nM or less, 1 nM or less. For example, the YTHDF2 inhibitor may show high binding activity towards YTH domain with Kd of 10000 nM or less, 1000 nM or less, 100 nM or less, 10 nM or less, 1 nM or less.

Such an attenuating agent may comprise or be a polypeptide. In some cases, such an attenuating agent may comprise or be a nucleic acid molecule. For example, such an attenuating agent may comprise an antibody or a derivative thereof, an antibody-drug conjugate, a fusion protein, and/or an antisense molecule.

In some cases, the agent capable of attenuating the expression and/or activity of YTHDF2 may comprise one or more of the following: a ubiquitin, a PROTAC, a dsRNA, a siRNA, a shRNA, an aptamer, and a gRNA. The agent (e.g., nucleic acid molecule or protein) may be naturally occurring or modified. For example, the RNAs or DNAs may be modified to be nuclease resistant.

In some cases, the agent capable of attenuating the expression and/or activity of YTHDF2 may comprise a mutant or a variant of YTHDF2 protein capable of attenuating the activity of an endogenous YTHDF2. In some cases, the agent capable of attenuating the expression and/or activity of YTHDF2 may comprise a nucleic acid molecule encoding for the mutant or variant of YTHDF2 protein.

For example, the agent capable of attenuating the expression and/or activity of YTHDF2 may comprise a dominant negative YTHDF2, or a nucleic acid molecule encoding said dominant negative YTHDF2. A dominant negative YTHDF2 may be a mutant or variant YTHDF2 protein, or the gene encoding the mutant or variant protein, that substantially prevents a corresponding YTHDF2 protein having wild-type function from performing the wild-type function. The wild-type function may comprise an activity of recognizing, binding to, and/or modifying m⁶A RNA.

Dominant negative gene products can be of many forms, including truncations, full length proteins with point mutations or fragments thereof, or fusions of full length wild type or mutant proteins or fragments thereof with other proteins. The level of inhibition observed can be very low. For example, it may require a large excess of the dominant negative protein compared to the functional protein or proteins involved in a process in order to see an effect. It may be difficult to see effects under normal biological assay conditions. In one embodiment, a dominant negative YTHDF2 may not be able to recognize, bind, and/or modify m⁶A RNA.

In some cases, the expression and/or activity of YTHDF2 may be attenuated by genetic manipulation. For example, the immune cells may have been subjected to a modification that results in a complete or partial deletion, a complete or partial replacement and/or reduced expression of a gene expressing YTHDF2.

For example, in a population of immune cells, one or more cells have been subjected to a modification that results in a complete or partial deletion, a complete or partial replacement and/or reduced expression of a gene expressing YTHDF2.

For example, such a modification may comprise homologous recombination, through the use of a nucleic acid molecule (e.g., vector) containing at least a portion of a Ythdf2 gene into which a deletion, addition or substitution has been introduced to thereby alter, e.g., functionally disrupt, the Ythdf2 gene. The Ythdf2 gene can be a human gene, or a nonhuman homologue of a human Ythdf2 gene. For example, a mouse Ythdf2 gene can be used to construct a homologous recombination vector suitable for altering an endogenous Ythdf2 gene, respectively, in the mouse genome. In some embodiments, the vector may be designed such that, upon homologous recombination, the endogenous Ythdf2 gene is functionally disrupted (i.e., no longer encodes a functional protein; also referred to as a “knock out” vector). Alternatively, the vector can be designed such that, upon homologous recombination, the endogenous Ythdf2 gene is mutated or otherwise altered but still encodes functional protein (e.g., the upstream regulatory region can be altered to thereby alter the expression of the endogenous Ythdf2 protein). In the homologous recombination vector, the altered portion of the Ythdf2 gene may be flanked at its 5′ and 3′ ends by additional nucleic acid of the Ythdf2 gene to allow for homologous recombination to occur between the exogenous Ythdf2 gene carried by the vector and an endogenous Ythdf2 gene a cell (e.g., an immune cell). The additional flanking Ythdf2 nucleic acid may be of sufficient length for successful homologous recombination with the endogenous gene. Typically, several kilobases of flanking DNA (both at the 5′ and 3′ ends) may be included in the vector. The vector may be introduced into the immune cells (e.g., by electroporation) and cells in which the introduced Ythdf2 gene has homologously recombined with the endogenous Ythdf2 gene may be selected.

In some cases, the modification is not applied directly to the immune cell (e.g., immune effector cell, such as T cell) itself, instead, the immune cell may be derived from (e.g., differentiated from, as a progeny of, etc.) a cell (e.g., a progenitor of an immune cell) or an organism that has been subjected to modifications resulting in a complete or partial deletion, a complete or partial replacement and/or reduced expression of a gene expressing YTHDF2 in said cell or said organism.

Such modified cell (e.g., immune cell or its progenitors) or organism (e.g., a transgenic nonhuman animal) may contain selected systems which allow for regulated expression and/or regulated deletion of a gene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355.

In some cases, the agent capable of attenuating the expression and/or activity of YTHDF2 may comprise or be (1) a gene editing system targeted to one or more sites within the gene encoding YTHDF2, or its regulatory elements, e.g., Ythdf2 or its regulatory elements; (2) nucleic acid encoding one or more components of said gene editing system; or (3) combinations thereof.

For example, the gene editing system may be selected from the group consisting of: a CRISPR/Cas9 system, a zinc finger nuclease system, a TALEN system and a meganuclease system.

The CRISPR/Cas system may be used in gene editing (silencing, enhancing or changing specific genes) in eukaryotes such as mice or primates. This is accomplished by, for example, introducing into the eukaryotic cell a plasmid containing a specifically designed CRISPR and one or more appropriate Cas. The CRISPR sequence, sometimes called a CRISPR locus, comprises alternating repeats and spacers. In a naturally-occurring CRISPR, the spacers usually comprise sequences foreign to the bacterium such as a plasmid or phage sequence; in an exemplary YTHDF2 CRISPR/Cas system, the spacers may be derived from the Ythdf2 gene sequence, or a sequence of its regulatory elements.

RNA from the CRISPR locus is constitutively expressed and processed into small RNAs. These comprise a spacer flanked by a repeat sequence. The RNAs guide other Cas proteins to silence exogenous genetic elements at the RNA or DNA level. Horvath et al. (2010) Science 327: 167-170; Makarova et al. (2006) Biology Direct 1: 7. The spacers thus serve as templates for RNA molecules, analogously to siRNAs. Pennisi (2013) Science 341: 833-836.

A CRISPR system may rely on the protein Cas9, which is a nuclease with two active cutting sites, one for each strand of the double helix. Combining Cas9 and modified CRISPR locus RNA can be used in a system for gene editing.

For example, the CRISPR/Cas system can be used to modify, e.g., delete one or more nucleic acids of the Ythdf2 gene, e.g., the Ythdf2 gene regulatory element, or introduce a premature stop which thus decreases expression of a functional YTHDF2. The CRISPR/Cas system can alternatively be used like RNA interference, turning off the Ythdf2 gene in a reversible fashion. In a mammalian cell, for example, the RNA can guide the Cas protein to a Ythdf2 promoter, sterically blocking RNA polymerases.

CRISPR/Cas systems for gene editing in eukaryotic cells typically involve (1) a guide RNA molecule (gRNA) comprising a targeting sequence (which is capable of hybridizing to the genomic DNA target sequence), and sequence which is capable of binding to a Cas, e.g., Cas9 enzyme, and (2) a Cas, e.g., Cas9, protein. The targeting sequence and the sequence which is capable of binding to a Cas, e.g., Cas9 enzyme, may be disposed on the same or different molecules. If disposed on different molecules, each may include a hybridization domain which allows the molecules to associate, e.g., through hybridization.

Artificial CRISPR/Cas systems can be generated which attenuate the activity and/or expression of YTHDF2, using technology known in the art, e.g., that are described in U.S. Publication No. 20140068797, WO2015/048577, and Cong (2013) Science 339: 819-823. Other artificial CRISPR/Cas systems that are known in the art may also be generated which inhibit YTHDF2, e.g., that described in Tsai (2014) Nature Biotechnol., 32:6 569-576, U.S. Pat. Nos. 8,871,445; 8,865,406; 8,795,965; 8,771,945; and 8,697,359, the contents of which are hereby incorporated by reference in their entirety. Such systems can be generated which inhibit YTHDF2, by, for example, engineering a CRISPR/Cas system to include a gRNA molecule comprising a targeting sequence that hybridizes to a sequence of a Ythdf2 gene. For example, the gRNA may comprise a targeting sequence which is fully complementarity to 15-25 nucleotides, e.g., 20 nucleotides, of a Ythdf2 gene. In some cases, the 15-25 nucleotides, e.g., 20 nucleotides, of a Ythdf2 gene, may be disposed immediately 5′ to a protospacer adjacent motif (PAM) sequence recognized by the Cas protein of the CRISPR/Cas system (e.g., where the system comprises a S. pyogenes Cas9 protein, the PAM sequence comprises NGG, where N can be any of A, T, G or C).

Foreign DNA can be introduced into the cell along with the CRISPR/Cas system, e.g., DNA encoding a CAR, e.g., as described herein; depending on the sequences of the foreign DNA and chromosomal sequence, this process can be used to integrate the DNA encoding the CAR, e.g., as described herein, at or near the site targeted by the CRISPR/Cas system. Such integration may lead to the expression of the CAR as well as disruption of the Ythdf2 gene.

In some cases, the gene editing system may comprise or is a CRISPR/Cas system comprising a gRNA molecule comprising a targeting sequence which hybridize to a target sequence of a Ythdf2 gene. The gene editing system may bind to a target sequence in an early exon or intron of a gene encoding YTHDF2. In some cases, the gene editing system may bind to a target sequence upstream of exon 4, e.g., in exon 1, exon 2, and/or exon 3 of a gene encoding YTHDF2. In some cases, the gene editing system may bind to a target sequence in a late exon or intron of a gene encoding YTHDF2. For example, the gene editing system may bind to a target sequence downstream of exon 3, e.g., in exon 4, exon 5, exon 6, exon 7, and/or exon 8 of a gene encoding YTHDF2.

In some cases, the gene editing system may bind to a target sequence in exon 1, exon 2, exon 3, exon 4, exon 5, exon 6, exon 7, and/or exon 8 of a gene encoding YTHDF2. In some embodiments, the targeting sequence is a targeting sequence as set forth in SEQ ID NO.17.

In some cases, TALEN gene editing systems may be used to attenuate the expression and/or activity of YTHDF2. TALENs are produced artificially by fusing a TAL effector DNA binding domain to a DNA cleavage domain. Transcription activator-like effects (TALEs) can be engineered to bind any desired DNA sequence, including a portion of the Ythdf2 gene. By combining an engineered TALE with a DNA cleavage domain, a restriction enzyme can be produced which is specific to any desired DNA sequence, including a Ythdf2 gene sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501. TALEs are proteins secreted by Xanthomonas bacteria. The DNA binding domain contains a repeated, highly conserved 33-34 amino acid sequence, with the exception of the 12^{th} and 13^{th} amino acids. These two positions are highly variable, showing a strong correlation with specific nucleotide recognition. They can thus be engineered to bind to a desired DNA sequence.

To produce a TALEN, a TALE protein is fused to a nuclease (N), which is, for example, a wild-type or mutated Fokl endonuclease. Several mutations to Fokl have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96.

The Fokl domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity. Miller et al. (2011) Nature Biotech. 29: 143-8.

A Ythdf2 gene TALEN can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN, e.g., DNA encoding a CAR, e.g., as described herein; depending on the sequences of the foreign DNA and chromosomal sequence, this process can be used to integrate the DNA encoding the CAR, e.g., as described herein, at or near the site targeted by the TALEN. As shown herein, in the examples, but without being bound by theory, such integration may lead to the expression of the CAR as well as disruption of the Ythdf2 gene.

TALENs specific to sequences in Ythdf2 gene, can be constructed using any method known in the art, including various schemes using modular components. Zhang et al. (2011) Nature Biotech. 29: 149-53; Geibler et al. (2011) PLoS ONE 6: e19509; U.S. Pat. Nos. 8,420,782; 8,470,973, the contents of which are hereby incorporated by reference in their entirety.

In some cases, zinc finger nuclease may be used to attenuate the expression and/or activity of YTHDF2. “ZFN” or “Zinc Finger Nuclease” refer to a zinc finger nuclease, an artificial nuclease which can be used to modify, e.g., delete one or more nucleic acids of, a desired nucleic acid sequence, e.g., Ythdf2 gene. Like a TALEN, a ZFN comprises a Fokl nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

A zinc finger is a small protein structural motif stabilized by one or more zinc ions. A zinc finger can comprise, for example, Cys₂His₂, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5.

Also like a TALEN, a ZFN can create a double-stranded break in the DNA, which can create a frame-shift mutation if improperly repaired, leading to a decrease in the expression and amount of Ythdf2 gene in a cell. ZFNs can also be used with homologous recombination to mutate the Ythdf2 gene, or to introduce nucleic acid encoding a CAR at a site at or near the targeted sequence. As discussed above, the nucleic acid encoding a CAR may be introduced as part of a template DNA.

ZFNs specific to sequences in the Ythdf2 gene can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; and Guo et al. (2010) J. Mol. Biol. 400: 96; U.S. Patent Publication 2011/0158957; and U.S. Patent Publication 2012/0060230, the contents of which are hereby incorporated by reference in their entirety. The ZFN gene editing system may also comprise nucleic acid encoding one or more components of the ZFN gene editing system, e.g., a ZFN gene editing system targeted to Ythdf2 gene.

In some cases, double stranded RNA (“dsRNA”), e.g., siRNA or shRNA can be used to attenuate Ythdf2, or as the YTHDF2 attenuating agent. Also contemplated by the present application are the uses of nucleic acid encoding said dsRNA Ythdf2 gene attenuating agent.

In some cases, the YTHDF2 attenuating agent is a nucleic acid, e.g., a dsRNA, e.g., a siRNA or shRNA specific for nucleic acid encoding YTHDF2, e.g., genomic DNA or mRNA encoding YTHDF2.

The present application provides a composition comprising a dsRNA, e.g., a siRNA or shRNA, comprising at least 15 contiguous nucleotides, e.g., 15, 16, 17, 18, 19, 20, 21, 22, 23, 24 or 25 contiguous nucleotides, e.g., 21 contiguous nucleotides, which are complementary (e.g., 100% complementary) to a sequence of a Ythdf2 gene nucleic acid sequence (e.g., genomic DNA or mRNA encoding YTHDF2). The at least 15 contiguous nucleotides, e.g., 15, 16, 17, 18, 19, 20, 21, 22, 23, 24 or 25 contiguous nucleotides, e.g., 21 contiguous nucleotides, may include contiguous nucleotides of a target sequence of shRNA or nucleic acid encoding YTHDF2 shRNA. It is understood that some of the target sequences and/or shRNA molecules are presented as DNA, but the dsRNA agents targeting these sequences or comprising these sequences can be RNA, or any nucleotide, modified nucleotide or substitute disclosed herein and/or known in the art, provided that the molecule can still mediate RNA interference.

Accordingly, in some cases, the agent capable of attenuating the expression and/or activity of YTHDF2 may comprise or be an siRNA or shRNA specific for Ythdf2, or nucleic acid encoding said siRNA or shRNA. In some embodiments, the siRNA or shRNA comprises a sequence complementary to a sequence of a Ythdf2 mRNA.

Cancer/Tumor Associated Antigens

In the present application, cancer associated antigens may be expressed on the surface of cancer cells. In some cases, the cancer associated antigens themselves may be intracellular, however, a fragment of such antigen (peptide) may be presented on the surface of the cancer cells by MHC (major histocompatibility complex). Examples of cancer/tumor associated antigens may include e.g., EGFR, HER2/neu, HER3, HER4, Ep-CAM, CEA, TrAIL, TRAIL receptor 1, TRAIL receptor 2, lymphotoxin-beta receptor, CCR4, CD19, CD20, CD22, CD28, CD33, CD40, CD80, CSF-1R, CTLA-4, fibroblast activation protein (FAP), hepsin, chondroitin proteoglycan sulfate associated with melanoma (MCSP), prostate specific membrane antigen (PSMA), VEGF receptor 1, VEGF receptor 2, IGF-1R, TSLP-R, TIE-1, TIE-2, TNF-alpha, similar weak apoptosis inducer to TNF (TWEAK), IL-1R, preferably EGFR, HER2/neu, CEA, CD20 and/or IGF-1R.

Pharmaceutically Acceptable Excipient

The composition of the present application may comprise one or more pharmaceutically acceptable excipients. Such pharmaceutically acceptable excipient may include any inactive material that is combined with one or more active ingredient (e.g., the modified cell or attenuating agent) of the present application.

For example, the pharmaceutically acceptable excipient may include one or more of the following: a solvent, a penetration enhancing agent, an antioxidant, a thickener, an ointment base, a protective, an adsorbent, a demulcent, an emollient, a preservative, a moisturizer, a buffer, an adjuvant, a bioavailability enhancer, a carrier, a glidant, a sweetening agent, a diluent, a dye/colorant, a flavor enhancer, a solubilizer (including surfactants), a wetting agent, a dispersing agent, a suspending agent, a stabilizer and/or an isotonic agent.

Combination Therapy

The modified cell (e.g., modified immune cell), the YTHDF2 attenuating agent, the composition and/or the method of the present application may be used in combination with one or more additional active ingredients or therapies (also referred to herein as the second active ingredient).

For example, the composition may comprise one or more of the additional active ingredients. In some cases, the modified cell (e.g., modified immune cell) may comprise an additional active ingredient, or may be administered in combination with an additional active ingredient or therapy.

The additional active ingredient or therapy may be administered prior to, concurrent with, or after the administration of the modified cell (e.g., modified immune cell), the composition, the YTHDF2 attenuating agent, and/or the method of the present application.

In some cases, the additional active ingredient may be comprised in the same package or the same container as the modified cell (e.g., modified immune cell) and/or the YTHDF2 attenuating agent of the present application. In some cases, the additional active ingredient may be contained in a separate container, for example, the additional active ingredient may be contained in a container different from that containing the modified cell (e.g., modified immune cell) and/or the YTHDF2 attenuating agent of the present application. In some cases, the additional active ingredient is not in direct contact with (e.g., does not mix with) the modified cell (e.g., modified immune cell) and/or the YTHDF2 attenuating agent of the present application, even though they may be present in the same container, or in the same package.

The additional active ingredient may be an anti-cancer agent. For example, the additional active ingredient may comprise a cancer immunotherapy. In some cases, the additional active ingredient may comprise an immune checkpoint attenuating agent. In some embodiments, the additional active ingredient may comprise an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO attenuating agent. For example, the additional active ingredient may comprise pembrolizumab, nivolumab, cemiplimab, atezolizumab, avelumab, durvalumab, ipilimumab, and/or any anti-cancer agent comprising one or more antigen binding portion of any of the foregoing.

In Vivo Method, In Vitro Method, Ex Vivo Method

The present application provides methods of increasing the activity and/or immune response of immune cells (e.g., immune effector cells) e.g., a cell expressing a CAR as described herein, e.g., a CAR 20-expressing cell, comprising a step of attenuating the expression and/or activity of YTHDF2 in said cell. The method may comprise reducing or eliminating the function or expression of YTHDF2.

For example, the method may comprise contacting said cells with a YTHDF2 attenuating agent as described herein. The contacting may be done ex vivo. In some cases, the contacting may be done in vivo. In some cases, the contacting may be done prior to, simultaneously with, or after said cells are modified to express e.g., a CAR or a TCR, e.g., as described herein.

The present application may provide a method, e.g., a method described above, comprising a step of introducing into the cell a gene editing system, e.g., a CRISPR/Cas gene editing system which targets the Ythdf2 gene, e.g., a CRISPR/Cas system comprising a gRNA which has a targeting sequence complementary to a target sequence of the Ythdf2 gene. In some cases, the CRISPR/Cas system may be introduced into said cell as a ribonuclear protein complex of gRNA and Cas enzyme, e.g., is introduced via electroporation. For example, the method may comprise introducing nucleic acid molecules encoding one or more of the components of the CRISPR/Cas system into said cell. In some cases, the nucleic acid may be disposed on the vector encoding a CAR, e.g., a CAR as described herein.

In some cases, the method may comprise a step of introducing into the cell an attenuating dsRNA, e.g., a shRNA or siRNA, which targets the Ythdf2 gene. For example, the method may comprise introducing into said cell nucleic acid encoding an attenuating dsRNA, e.g., a shRNA or siRNA, which targets the Ythdf2 gene. In some cases, the nucleic acid may be disposed on the vector encoding a CAR, e.g., a CAR as described herein.

Disease, Disorder or Condition

The cells, methods and compositions of the present application may be used for preventing, ameliorating, and/or treating a disease, disorder or condition, such as a disease, disorder or condition associated with the expression of a cancer/tumor associated antigen as described herein.

For example, the disease, disorder or condition may be cancer.

In some cases, the cancer may be selected from the group consisting of a hematological cancer, a lymphoma, and a solid tumor.

In some cases, the cancer may be selected from the group consisting of melanoma, colon cancer, pancreatic cancer, breast cancer, lung cancer, and liver cancer.

Subject

The modified immune cells, the YTHDF2 attenuating agent, the composition, and/or the method of the present application may be administered to a subject in need thereof.

In some cases, the subject may be a cancer patient. For example, the subject may be a patient of a cancer selected from the group consisting of a hematological cancer, a lymphoma, and a solid tumor. In some cases, the subject may be a patient of a cancer selected from the group consisting of melanoma, colon cancer, pancreatic cancer, breast cancer, lung cancer, and liver cancer.

In some cases, the subject may have received, is receiving, and/or will receive an additional therapy. The additional therapy may be an anti-cancer treatment.

In some cases, the anti-cancer treatment may comprise a cancer immunotherapy. For example, the anti-cancer treatment may comprise or is an immune checkpoint attenuating agent. In some cases, the anti-cancer treatment may comprise an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO attenuating agent. In some cases, the anti-cancer treatment may comprise pembrolizumab, nivolumab, cemiplimab, atezolizumab, avelumab, durvalumab, and/or ipilimumab.

Activating an Immune Cell and Enhancing an Immune Response

The YTHDF2 attenuating agent, the modified immune cells, the composition, and the method of the present application may be used for activating an immune cell, and/or for enhancing an immune response, e.g. an anti-tumor immune response.

For example, an activated immune cell may have increased ability (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) to kill tumor cells or control tumor growth in vivo.

In some cases, in a population of immune cells, an increased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) proliferation of CD4⁺ T cells may be observed. In some cases, in a population of immune cells, an increased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) proliferation of CD8⁺ T cells may be observed.

In some cases, an enhanced anti-tumor immune response may be revealed by an increase (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of the number of CD8⁺ cytotoxic T cells in or surrounding the site of a tumor.

In some cases, an enhanced anti-tumor immune response may be revealed by an increase (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of the number of tumor infiltrating CD8⁺ T cells.

In some cases, increased activity of an immune cell (e.g. T cell) may be revealed by increased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) cytokine (e.g., IFN-γ, and/or IL-2) production by the immune cells.

In some cases, an increased activity of immune cells or an enhanced immune response may be revealed by delayed and/or reversed (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) exhaustion of an immune cells, such as delayed and/or reversed (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) exhaustion of CD8⁺ T cells.

For example, the increased activity of immune cells or an enhanced immune response may be revealed by an increased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) expression of TCF-1 and/or TCF-7. The increased expression may either be characterized by an increased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) amount/level of TCF-1 and/or TCF-7 in/on the cells, or be characterized by an increased number/percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of cells that express TCF-1 and/or TCF-7 among a population of immune cells (e.g., a population of immune effector cells, such as a population of T cells).

In some cases, an increased activity of immune cells or an enhanced immune response may be revealed by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) expression of T-bet. The decreased expression may either be characterized by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) amount/level of T-bet in/on the cells, or be characterized by a decreased number/percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of cells that express T-bet among a population of immune cells (e.g., a population of immune effector cells, such as a population of T cells).

In some cases, an increased activity of immune cells or an enhanced immune response may be revealed by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) expression of Eomesodermin (Eomes). The decreased expression may either be characterized by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) amount/level of Eomes in/on the cells, or be characterized by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) number/percentage of cells that express Eomes among a population of immune cells (e.g., a population of immune effector cells, such as a population of T cells).

In some cases, an increased activity of immune cells or an enhanced immune response may be revealed by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) expression of PD-1. The decreased expression may either be characterized by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) amount/level of PD-1 in/on the cells, or be characterized by a decreased number/percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of cells that express PD-1 among a population of immune cells (e.g., a population of immune effector cells, such as a population of T cells).

In some cases, an increased activity of immune cells or an enhanced immune response may be revealed by a decreased expression (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of Tim-3. The decreased expression may either be characterized by a decreased amount/level (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of Tim-3 in/on the cells, or be characterized by a decreased number/percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of cells that express Tim-3 among a population of immune cells (e.g., a population of immune effector cells, such as a population of T cells).

In some cases, an increased activity of immune cells or an enhanced immune response may be revealed by an increased number and/or percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of CD45⁺ cells (e.g. CD45⁺CD4⁺ cells' or CD45⁺CD8⁺ cells) within a population of immune cells (e.g., a population of immune effector cells, such as a population of T cells).

In some cases, an increased activity of immune cells or an enhanced immune response may be revealed by an increased number and/or percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of PD1⁻TCF1⁺ cells within a population of immune cells (e.g., a population of immune effector cells, such as a population of T cells).

In some cases, an increased activity of immune cells or an enhanced immune response may be revealed by an increased number and/or percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of PD1⁻TCF1⁺CD62L⁻cells within a population of immune cells (e.g., a population of immune effector cells, such as a population of T cells).

In some cases, an increased activity of immune cells or an enhanced immune response may be revealed by an increased number and/or percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of PD1⁻TCF1⁺CD62L⁺cells within a population of immune cells (e.g., a population of immune effector cells, such as a population of T cells).

In some cases, an increased activity of immune cells or an enhanced immune response may be revealed by an increased number and/or percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of Tim3⁻TCF1⁺ cells within a population of immune cells (e.g., a population of immune effector cells, such as a population of T cells).

In some cases, an increased activity of immune cells or an enhanced immune response may be revealed by an increased number and/or percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of IFN-γ⁺CD8⁺ cells within a population of immune cells (e.g., a population of immune effector cells, such as a population of T cells).

In some cases, an increased activity of immune cells or an enhanced immune response may be revealed by an increased number and/or percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of IFN-γ⁺IL-2⁺ cells within a population of immune cells (e.g., a population of immune effector cells, such as a population of T cells).

In some cases, an increased activity of immune cells or an enhanced immune response may be revealed by a decreased number and/or percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 1.5 fold, at least about 2 folds, at least about 2.5 folds, at least about 3 folds, at least about 3.5 fold, or more) of Tim3⁺ TCF1⁻ cells within a population of immune cells (e.g., a population of immune effector cells, such as a population of T cells).

### EXAMPLES

The following examples are set forth so as to provide those of ordinary skill in the art with a complete disclosure and description of how to make and use the present application, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

Materials and Methods

Mice

Ythdf2^{flox/flox} mice were generated as previously described, CD4-Cre transgenic mice were generated by standard procedures. Mice used for experiments were further backcrossed to C57BL/6J for two generations. To ensure the comparability in genetic background, mice were maintained by crossing CD4^{cre}Ythdf2^{flox/flox} and Ythdf2^{flox/flox}. CD4^{cre}Ythdf2^{flox/flox} or their littermates control WT mice were used in all experiments. Littermates were co-housing during experiments to reduce variants in their microbiome and environment. Primers used for genotyping of Ythdf2^{flox/flox} mice: GAACGGTATTGTCGGTATTGTCA (SEQ ID NO.1) and AGACCACTCCAACACAGAACTT (SEQ ID NO.2) primers used for genotyping of CD4-Cre: GTTCTTTGTATATATTGAATGTTAGCC(SEQ ID NO.3) TATGCTCTAAGGACAAGAATTGACA (SEQ ID NO.4) and CTT TGC AGA GGG CTA ACA GC (SEQ ID NO.5). CD45.1 OTI mice were purchased from Jackson Laboratory. CD45.1 OTI CD4^{cre}DF2^{flox/flox} mice were generated in house. All mice were used at 6-12 weeks of age. All the mice were maintained under specific pathogen-free conditions and used in accordance with the animal experimental guidelines set by the Institutional Animal Care and Use Committee of Tsinghua University.

Cell Lines

B16-OVA is an OVA-transfected clone derived from the murine melanoma cell line B16. mB16-zsGreen-OTIp (B16-OZ) was selected for a single clone after being transduced by lentivirus expressing zsGreen-OTIp (SIINFEKL). MC38 is a murine colon adenocarcinoma cell line. E. G7 is an OVA-transfected clone derived from the mouse lymphoma cell line EL4. E. G7 is culture with RPMI 1640 (Thermo) with 10% FBS and 1% penicillin-streptomycin supplemented with 0.1 M HEPES buffer, 0.1 mM non-essential amino acid at 37° C. in 5% CO2 and other cells were maintained in DMEM (Thermo) with 10% FBS and 1% penicillin-streptomycin supplemented with 2 mM L-glutamine, 0.1 M HEPES buffer, 0.1 mM non-essential amino acid at 37° C. in 5% CO2.

Lenti-X 293 cell line was purchased from Clontech (Mountain View, Calif.). Raji cells were kindly provided by Stem Cell Bank, Chinese Academy of Sciences (Shanghai, China). Lenti-X 293 was cultured in DMEM. Raji was maintained in RPMI-1640. All cell culture mediums were supplemented with 10% heat-inactivated fetal bovine serum (Gibco), 2 mmol/L L-glutamine, 100 units/mL penicillin, and 100 μg/mL streptomycin.

Primary Cell Cultures

Single-cell suspension of T cells were cultured in RPMI-1640 medium containing 10% fetal bovine serum, stimulated with 2 μg/mL anti-CD3 and 0.5 μg/mL anti-CD28 (Invitrogen), supplemented with 55 uM 2-mercaptoethanol, 0.1 M HEPES buffer, 0.1 mM non-essential amino acid at 37° C. in 5% CO₂.

In Vitro Tolerance Induction

CD8⁺ T cells from CD4^{cre}DF2^{f/f} mice were isolated with EasySep™ Mouse CD8⁺ T Cell Isolation Kit (Cat.19853). 5×10⁵/mL CD8⁺ T cells were stimulated with 2 μg/mL anti-CD3, 0.5 μg/mL anti-CD28 (Invitrogen) and 10 ng/mL IL-2, in RPMI-1640 complete medium on day 0, 3, 5 for 24h. Then We removed the stimulation medium and substituted with complete medium supplemented with only IL-2 to rest the cells. After three rounds of stimulation, live cells were purified for flow cytometry.

Tumor Growth and Treatments

About 1×10⁶ B16-OVA or MC3 8 or E. G7 tumor cells were subcutaneously inoculated into the flank of mice. Tumor volumes were measured by length(a) and width(b) and calculated as tumor volume=ab²/2. Mice with tumor volumes less than 2000 mm³ are considered to be survival. For in vivo depletion experiments, 200 μg of anti-CD8 or anti-CD4 antibody was injected intraperitoneally three days after tumor inoculation. For anti-PD-L1 treatment, 1×10⁶ B16-OVA tumor cells were inoculated subcutaneously into the flank of mice. 100 μg anti-PD-L1 antibody or rat immunoglobulin were administered on day 9 after tumor inoculation. For adoptive transfer of T cells, Rag1^{-/-} or other recipient mice were inoculated with 5×10⁵ B16-OVA on day 0. On the same day, T cells were purified from CD45.1 OTI CD4^{cre}DF2^{f/f} or CD45.1 OTI DF2^{f/f} mice using T cell negative isolation Kit (Stemcell). 5×10⁶ T cells were intravenously injected into recipient mice.

Acute and Chronic Viral Infection

Mice were generally infected intraperitoneally with LCMV-Armstrong (2×10⁵ plaque-forming units (PFU)) or intravenously with LCMV-clone 13 (2×10⁶ PFU). Mice were infected at 6-10 weeks of age, and both sexes were included without randomization or blinding. Eight days after infection, mice were euthanized and splenocytes were assessed. Flow cytometry analysis of CD8⁺ T cells in the spleen was performed using LCMV-GP33-41-tetramer (GP33⁺) staining.

Flow Cytometry and Cell Sorting

For flow cytometric analysis and cell sorting in Examples 1-4 (where applicable), tumors, lymph nodes and spleens were collected from mice and digested with 0.26 U/mL Liberase TL and 0.25 mg/mL DNase I at 37° C. for 30 min. Samples were then filtered through a 70 μm cell strainer and washed twice with staining buffer. Cells were re-suspended in staining buffer (PBS with 2% FBS and 1 mM EDTA). Cells were incubated with Fc Block (clone 2.4G2) for 10 min. Specific antibodies were subsequently added and stained for 30 min on ice. OT-I specific T cells were stained using iTAg Tetramer/H-2KbOVA (SIINFEKL)(MBL). After a washing step, cells were either analyzed on a BD Fortessa (BD) or sorted by Aria IIIu (BD). Analysis of flow cytometry data was performed using Flowjo.

For flow cytometric analysis and cell sorting in Examples 5-8 (where applicable), single cell suspensions were incubated with anti-CD16/32 (anti-FcγRII/III, clone 2.4G2) for 10 min and then subsequently stained with conjugated antibodies. The fluorescently labeled monoclonal antibodies are listed as the following: anti-human CD3-FITC (OKT3) and anti-human CD19-APC (H1B19) are from Biolegend; Alexa Fluor 647 conjugated goat anti-mouse Fab antibodies are from Jackson ImmunoResearch Laboratories. Samples were analyzed on a Cytoflex (Beckman Coulter), and data were analyzed with FlowJo software (TreeStar, Inc.).

RNA-Seq

Tumor infiltrating CD8⁺ T cells from CD4^{cre}DF2^{f/f} or DF2^{f/f} mice on day 7 post tumor inoculation were sorted by flow cytometry. Gp33⁺ CD8⁺ T cells were isolated from the spleen of LCMV clone 13 infected CD4^{cre}DF2^{f/f} or DF2^{f/f} mice. Total RNA was extracted from T cells with TRIzol reagent (Invitrogen). RNA library was constructed with SMARTER Stranded Total RNA-seq Kit V2-Pico input (Clontech 634418)

m⁶A-Seq

Total RNA was isolated from T cells. Polyadenylated RNA was further enriched by using Dynabeads mRNA Purification Kit (Invitrogen). RNA samples were fragmented into about 100-nucleotide-long fragments with RNA fragmentation reagent (Thermo) at 94° C. for 45s. Fragmented RNA (100 ng mRNA or 5 μg total RNA) was used to perform m⁶A-IP following EpiMark N6-methyladenosine Enrichment Kit (NEB E1610S) protocol. RNA was enriched through RNA Clean&Concentration-5 (Zymo Research) and used for library generation with SMARTER Stranded Total RNA-seq Kit V2-Pico input (Clontech 634418). Sequencing was performed on an Illumina HiSeq4000 machine.

ATAC-Seq

Tumor infiltrating CD8⁺ T cells or in vitro-cultured tolerant T cells were lysed with ATAC-RSB buffer. The cell lysate was digested with Tn5 transposase on ice. Then the library construction was performed by using TruePrep DNA Library Prep Kit V2 for Illumina (Vazyme). Sequencing was performed on an Illumina HiSeq4000 machine.

Design and Production of CAR

The antigen-targeting region scFv (SEQ ID NO. 6) of the Chimeric Antigen Receptor (CAR) was derived from Rituximab. “20 CAR” (SEQ ID NO. 11) comprises the scFv derived from Rituximab, the intracellular signaling domains 41BB (SEQ ID NO. 7) and CD3 (SEQ ID NO. 8), the CD8a hinge domain (SEQ ID NO. 9), and the 41BB transmembrane domain (SEQ ID NO. 10), wherein the scFv is linked to the intracellular signaling domains by the CD8a hinge domain and the 41BB transmembrane domain. YTHDF2 (SEQ ID NO. 15) was linked to CD3 (SEQ ID NO. 8) by P2A peptide (SEQ ID NO. 16) to generate 20-YTHDF2 (SEQ ID NO. 14).

20 CAR coding DNA (SEQ ID NO. 12) and 20-YTHDF2 CAR coding DNA (SEQ ID NO. 13) were cloned into pCDH-EF1-MSC vector backbone (Palo Alto, Calif., USA) to generate lentiviral transfer vector. Lentiviruses were produced by transient transfection of Lenti-X 293 cells with the vector plasmids generated. Supernatant containing lentivirus particles was collected 48 and 72h post-transfection, and then concentrated by ultracentrifugation at 25000 rpm at 4° C. (Beckman). The concentrated virus was slowly dissolved in complete RPMI-1640 medium for 4-16h. Viral titer was determined at indicated volume by flow cytometry analysis of transduced Lenti-X 293 cells.

Manufacture of CAR-T Cells

Peripheral blood mononuclear cells (PBMCs) were provided by Shanghai Longyao Biotechnology Co., Ltd. (Shanghai, China) and were purified by negative selection using an EasySep™ Human T Cell Isolation Kit (Stem cell). Purified T cells were seeded into 96-well plate and stimulated with anti-CD3 and anti-CD28 antibodies for 72 hours. Activated T cells were then transduced with lentivirus encoding 20 CAR or 20-YTHDF2 CAR at a final multiplicity of infection (MOI) of 10. For 20-YTHDF2-Knockout CAR-T cells (referred to herein as 20-YTHDF2-KO), 20 CAR-T cells were collected and washed twice with PBS and resuspended in P3 primary cell solution (Lonza). Alt-R crRNA (SEQ ID NO: 17) (90 pmol) and Alt-R tracrRNA (SEQ ID NO: 18) (45 pmol) (IDT) was reconstituted with Nuclease-free Duplex Buffer (IDT), Oligos were annealed by heating at 95° C. for 5 min in PCR thermocycler and the mix was slowly cooled to room temperature. CrRNA-tracrRNA duplexes and Cas9 Protein V3 (50 μg) (IDT) were gently mix by pipetting up and down and incubating at room temperature for at least 15 min. T cells were mixed and incubated with 5 μl RNP at room temperature for 2 min. The cell/RNP mix were electroporated using a 4D nucleofector (4D-Nucleofector Core Unit: Lonza), prewarmed T cell media was used to transfer transfected cells in 96-well plates. During in vitro expansion, CAR-T cells were stimulated weekly by irradiated Raji cells. CAR-T cells were cultured in RPMI-1640 medium with 200 IU/mL IL-2 and 4 ng/mL IL-21.

In Vitro Tumor Cell Killing Assay

A total of 1×10⁵ CAR-T cells were incubated with Raji cells at different effector: target (E:T) ratios (e.g., 1:1 or 1:2) in 96-well plate. 24 h after plating, cells were harvested and analyzed by flow cytometry. Anti-CD3 and anti-CD19 were used to distinguish CAR-T cells and tumor cells, respectively.

### Example 1 Generation of Knock Out Mice

Ythdf2^{flox/flox} mice were generated as previously described and maintained on a C57BL/6 background. For some embodiments, mice were crossed to OT-I TCR transgenic mice or mice that expressed Cre recombinase under the control of the Cd4 gene regulatory elements (Cd4Cre). In some embodiments, CD4^{cre}Ythdf2^{flox/flox} mice were crossed to OT-I TCR transgenic mice.

### Example 2 Evaluation of T Cell Functions

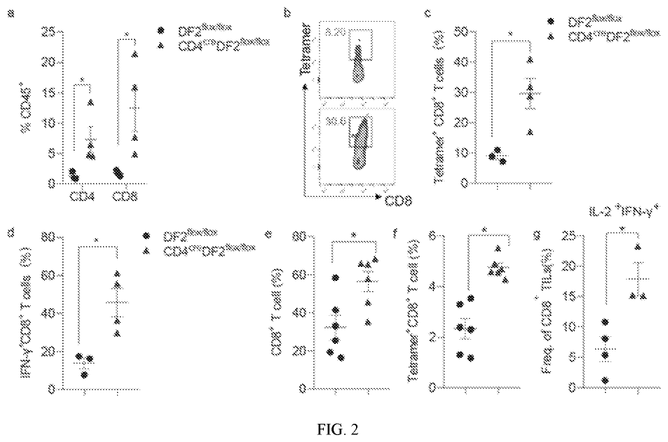

To determine whether neoantigen-specific CD8⁺ T cell responses are generated in E. G7 tumors, the frequency of tumor infiltrating SIINFEKL MHC-I tetramer⁺ CD8⁺ T cells in CD4^{cre}Ythdf2^{flox/flox} (CD4^{cre}DF2^{f/f}) and Ythdf2^{flox/flox} (DF2^{f/f}) mice was analyzed. As shown in FIG. 2, the percentage of both CD4⁺ and CD8⁺ T cells were improved in CD4^{cre}Ythdf2^{flox/flox} mice (FIG. 2a), indicating the enhanced T cell infiltration in tumor microenvironment. In addition, while DF2^{f/f} mice failed to accumulate antigen-specific CD8⁺ T cells within the tumor, CD4^{cre}DF2^{f/f} mice showed substantially increased CD8⁺ T cells against tumor neoantigen in vivo compared with DF2^{f/f} mice (FIG. 2b-2c). To further investigate the function of tumor infiltrating T cells, tumor infiltrating T cells were stimulated with PMA and ionomycin and blocked with BFA for 2h. Cells producing IFN-γ was quantified and the percentage of IFN-γ⁺ CD8⁺ T cells was significantly increased in CD4^{cre}DF2^{f/f} mice than in control mice (FIG. 2d). Similar results were observed in B16 melanoma model (FIG. 2e-2g).

### Example 3 Evaluation of Anti-Tumor Effects

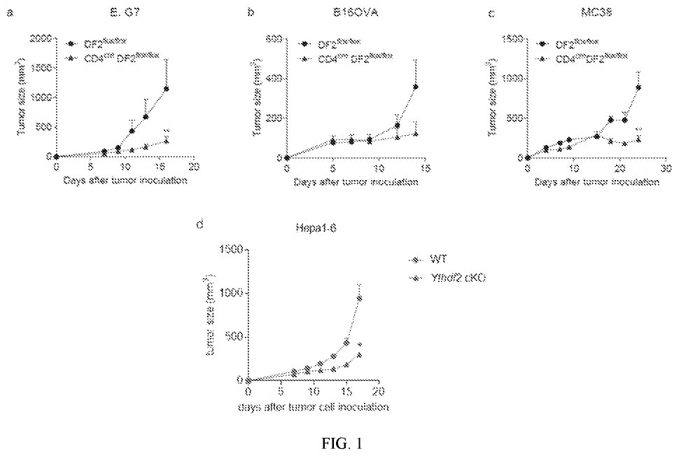

The m⁶A reader protein Ythdf2 conditional knock out mice (FIG. 1a) were inoculated with ovalbumin (OVA)-expressing lymphoma E. G7 cells subcutaneously along with WT control DF2^{flox/flox} mice. Compare to WT control mice, CD4^{cre}Ythdf2^{flox/flox} mice exhibited better tumor control and prolonged survival. These findings were also tested in OVA-expressing B16 melanoma model, MC38 cell colon carcinoma models and Hepa 1-6 cell hepatocellular carcinoma, which have been reported to have a broader neoantigen pool. A similar level of tumor inhibition was observed in CD4^{cre}Ythdf2^{flox/flox} mice comparing to that in WT control mice (FIGS. 1b-1d).

### Example 4 Reversion of T Cell Exhaustion

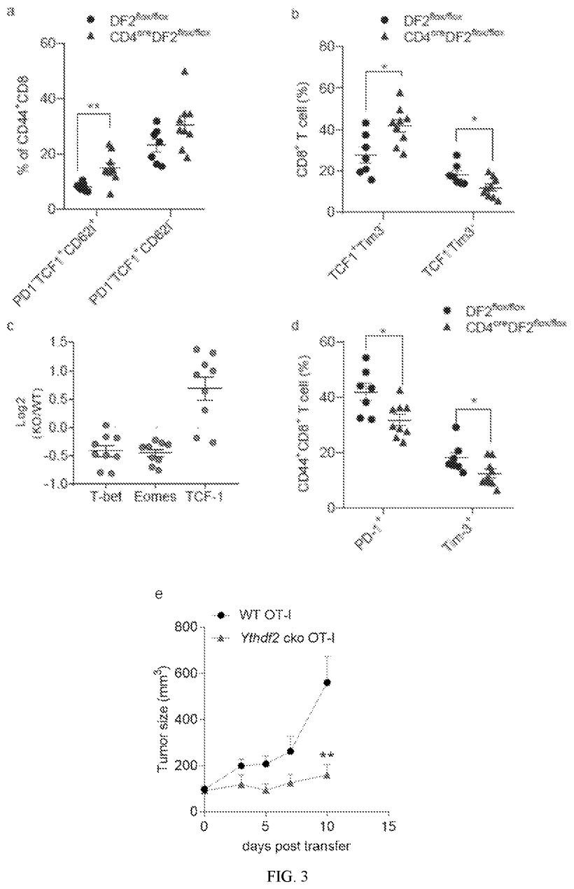

As can be seen from the results of the above Example, loss of Ythdf2 exhibits enhanced anti-tumor capacity. In addition, it was found that the tumor infiltrating CD8⁺ T cells were at different stage of exhaustion in Ythdf2 conditional knock out mice and in WT mice. Naïve T cells marked with PD-1⁻TCF1⁺CD621⁺ were accumulated in Ythdf2 conditional knock out mice (FIG. 3a). The exhausted T cells (TCF1⁻Tim3⁺ cells) were of higher density in the WT control mice (DF2^{flox/flox} mice), while the percentage of exhausted T cells progenitors (TCF1⁺ Tim3⁻) increased in the Ythdf2 conditional knock out mice (FIG. 3b). T cell factor-1 (TCF-1) critically regulates T cells development. Recent studies revealed that TCF-1 not only controls the early development of T cell fate determination, but is also involved in the process of T cell exhaustion.

It was found that TCF-1 expression was upregulated in Ythdf2 conditional knock out T cells by about two-fold comparing to that in WT T cells (FIG. 3c). PD-1 and Tim3 expression were also decreased in T cells of Ythdf2 conditional knock out mice comparing to that of WT mice (FIG. 3d).

5×10⁵ Wild-type (WT) OT-I (n=5) and Ythdf2 cKO OT-I (n=8) cells were adoptively transferred into B16-OVA bearing mice after 7 days post tumor inoculation. The tumor growth was monitored every other day. Ythdf2 conditional knock out T cells exhibited better tumor control and prolonged survival (FIG. 3e).

As for combination of YTHDF2 deficiency and immune checkpoint blockades, MC38 bearing WT control DF2^{f/f} mice and CD4^{cre}DF2^{f/f} mice with or without anti-PD-L1 antibody and anti-CD40 antibody were tested.

### Example 5 Expression of CARs

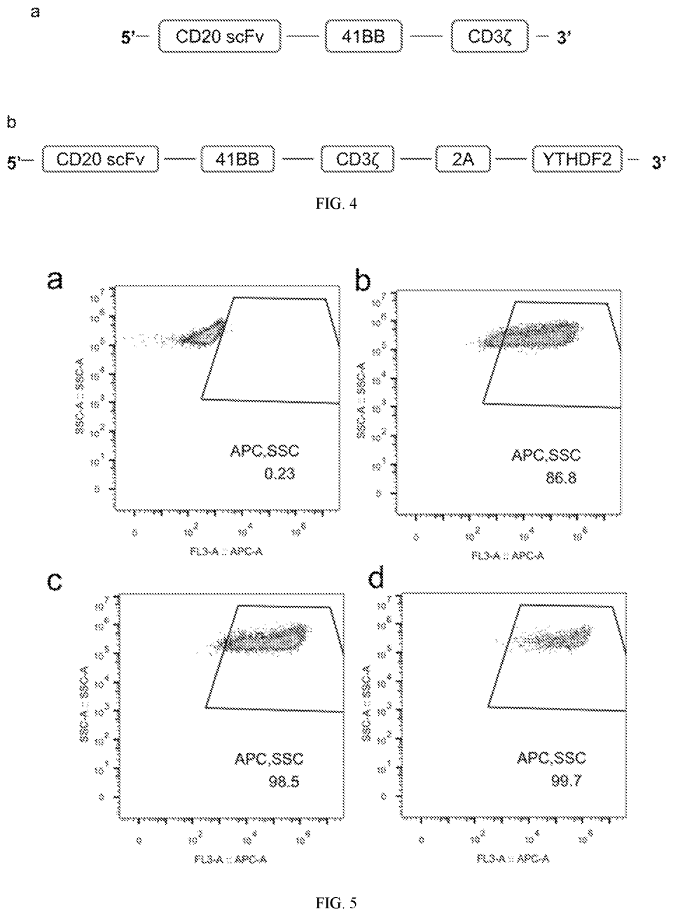

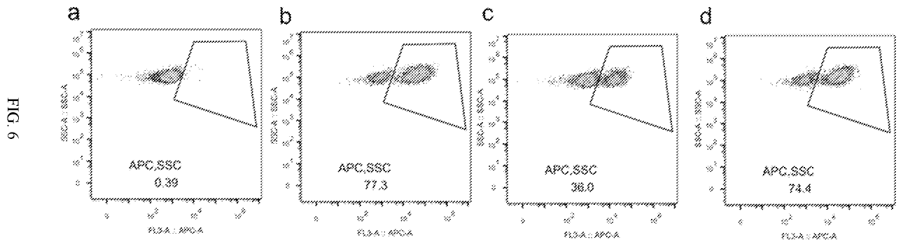

FIGS. 4a-4b illustrate the design of 20 CAR. 2×10⁵ Lenti-X 293 cells were infected with 0 μl (FIG. 5a), 0.33 μl (FIG. 5b), 1 μl (FIG. 5c), or 3 μl (FIG. 5d) of concentrated virus in the presence of 10 μg/ml polybrene in 24-well plate. 24 hours later, complete DMEM medium was added to the cells and further incubated at 37° C., 5% CO2. Virus titer was measured by flow cytometry analysis 48 hours later. It was found that 86.8% of Lenti-X 293 cells expressed the CAR, when only 0.33 μl of concentrated virus was used (FIG. 5b). In addition, the increased expression of CAR was dependent on the dose of the virus administered.

FIG. 8A illustrates the design of CLDN18.2 CARs.

### Example 6 Generation of CAR-T Cells

To evaluate whether the CARS would be expressed on the surface of T cells, human primary T cells were stimulated with 0.25 μg/ml anti-CD3 and 1 μg/ml anti-CD28 for 2 days, the cells were left to rest for 3 days, and then were infected by virus at a MOI of 10, to generate 20 CAR-T (expressing 20 CAR) and 20-YTHDF2-OE CAR-T (expressing 20-YTHDF2 CAR) cells. Further, 20 CAR-T cells were used to generate 20-YTHDF2-KO CAR-T cells by using CRISPR/Cas9 gene editing system to knock-out YTHDF2 therein. Five days later, the expression of CAR was examined by flow cytometry. As shown in FIG. 6, over 70% of the transfected T cells expressed 20 CAR (FIG. 6b for 20 CAR-T, and FIG. 6d for 20-YTHDF2-KO CAR-T), and over 30% of the transfected T cells expressed 20-YTHDF2 CAR (FIG. 6c for 20-YTHDF2-OE CAR-T cells). In addition, after stimulating the T cells weekly with irradiated Raji cells, the expression rate of CARs increased to about 100%.

An anti-human CLDN18.2 single-chain variable fragment (scFv)(SEQ ID NO:22) was linked to CD8 hinge Tm (SEQ ID NO:23), 4-1BB (SEQ ID NO:7) and CD3ζ (SEQ ID NO:8) to generate the CAR construct. YTHDF2 sgRNA (SEQ ID NO:24 for #5 and SEQ ID NO:25 for #6) or YTHDF2 (SEQ ID NO:15) was linked to CD3ζ via a porcine teschovirus-1 2A (P2A) (SEQ ID NO:16) peptide. The CAR coding DNA was cloned into the pCDH-MSC-EF1 vector backbone (SBI System Biosciences, Palo Alto, Calif.) to generate a lentiviral transfer vector. The lentivirus was produced using Lenti-X 293 T cells.

Peripheral blood mononuclear cells (PBMCs) were derived from cord blood provided by Shanghai Longyao Biotechnology Co., Ltd. (Shanghai, China) and were isolated using Ficoll-Paque density-gradient centrifugation. Total T cells were purified using an EasySep™ Human T Cell Isolation Kit (Stemcell). Purified T cells were seeded into 96-well plates and stimulated for 72 h with plate-bound anti-CD3 (0.25 μg/mL) and anti-CD28 (1 μg/mL) antibodies. Activated T cells were then transduced with lentivirus encoding the indicated CAR at a multiplicity of infection (MOI) of 10. During in vitro expansion, CAR-T cells were stimulated weekly with irradiated Raji cells (effector to target (E:T)=3:1). CAR-T cells were cultured in RPMI-1640 medium supplemented with 10% heat-inactivated FBS, 2 mmol/L-glutamine, 100 units/mL penicillin, and 100 μg/mL streptomycin, 50 IU/mL IL-2, and 4 ng/mL IL-21. CLDN18.2 CAR with YTHDF2 overexpression was constructed by lentivirus transfection and YTHDF2 knockout CAR was constructed by CRISPR/cas9 technology using YTHDF2 sgRNA electrorotation with CLDN18.2 CAR. Single-cell suspensions of cells were incubated with anti-CD16/32 (anti-FcgIII/II receptor, clone 2.4G2) for 10 min and then stained with conjugated Abs indicated. All fluorescently labeled monoclonal antibodies (mAbs) were purchased from Biolegend or eBioscience. Samples were analyzed on a Cytoflex flow cytometer (Beckman Coulter) and the data analyzed using FlowJo software V10 (TreeStar). After stimulation of irradiated Raji cells, the proportion of CAR-T could rise to nearly 100%. As shown in FIG. 8B, CLDN18.2 CAR-T (expressing CLDN18.2 CAR), CLDN18.2-YTHDF2-OE CAR-T (expressing CLDN18.2-YTHDF2 CAR), CLDN18.2-YTHDF2-KO CAR-T-#5 (expressing CLDN18.2 CAR and knocking-out YTHDF2 with sgRNA #5), CLDN18.2-YTHDF2-KO CAR-T-#6 (expressing CLDN18.2 CAR and knocking-out YTHDF2 with sgRNA #6), and control T cells are generated.

### Example 7 Tumor Cell Killing Effects of CAR-T Cells

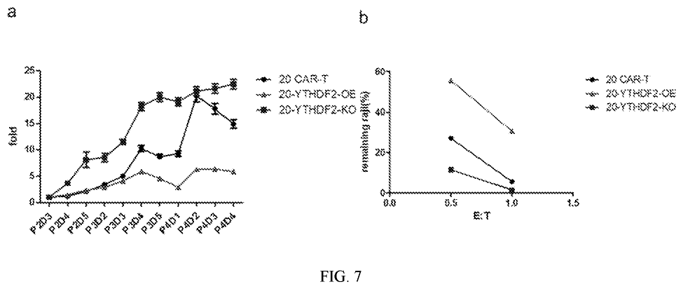

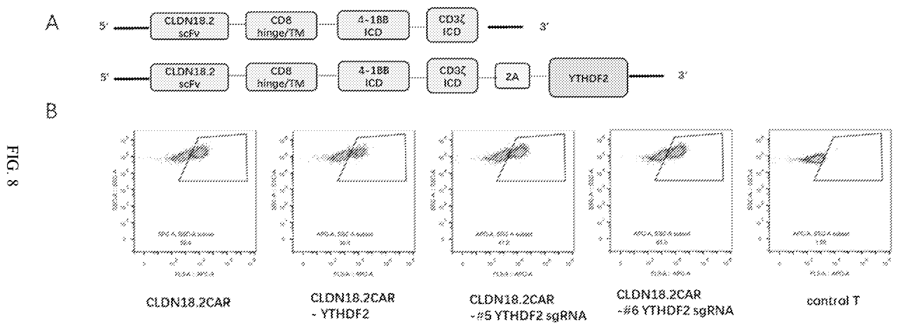

The proliferation of the 20 CAR-T, 20-YTHDF2-OE CAR-T, and 20-YTHDF2-KO CAR-T cells generated according to Example 6 was examined. Briefly, the cells were stimulated weekly with irradiated Raji cells. Cell counts were recorded, as revealed by trypan blue. 20-YTHDF2-KO CAR-T cells were observed to proliferate faster than 20 CAR-T cells, or 20-YTHDF2-OE CAR-T cells, especially in the later phases of the long-term culture, as shown in FIG. 7a.

Then, the tumor killing activity of the 20 CAR-T, 20-YTHDF2-OE CAR-T, and 20-YTHDF2-KO CAR-T cells generated according to Example 6 was examined, using the lymphoma cell line Raji as an example. Briefly, 1×10⁵ CAR-T cells were co-cultured with the Raji cells in triplicate at the various indicated effector: target (E:T) ratios. Their abilities to kill the tumor cells were determined by analyzing the remaining tumor cells (CD3⁻CD19⁺) with flow cytometry 24 hours later. As shown in FIG. 7b, 20-YTHDF2-KO CAR-T cells had significantly superior tumor killing activity, comparing to 20 CAR-T cells, and the YTHDF2 overexpressing 20-YTHDF2-OE CAR-T cells showed the lowest activity in killing tumor cells, the results obtained with an E:T ratio of 1:1 and 1:2 are similar.

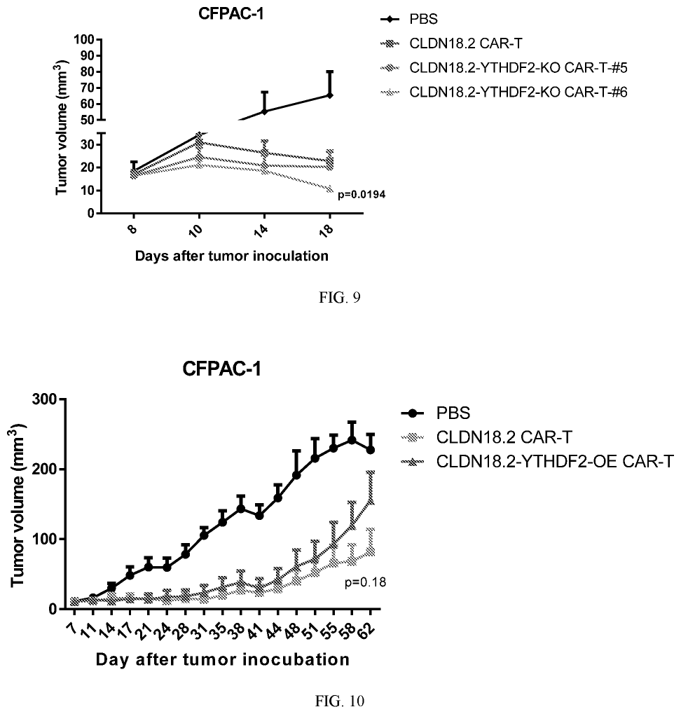

The antitumor effect of CLDN18.2 CARS with YTHDF2 knockout in CFAPC-1 in vivo was examined, as shown in FIG. 9. Female NOD/SCID/γ−/− (NSG) mice were purchased from Shanghai Model Organisms Center, Inc. (Shanghai, China) and were maintained under specific pathogen-free conditions. Animal care and use were in accordance with institutional and National Institutes of Health (NIH) protocols and guidelines. NSG mice (n=6) were subcutaneously inoculated with 2*10\hat{ }6 CFPAC-1 (pancreatic adenocarcinoma cell line). One week after tumor cell inoculation, the mice were randomly grouped and treated with PBS, 1*10\hat{ }7 CLDN18.2 CAR-T or 1*10\hat{ }7 CLDN18.2 CAR-T with YTHDF2 knockout (CLDN18.2-YTHDF2-KO CAR-T-#5 or CLDN18.2-YTHDF2-KO CAR-T-#6). Tumor volume were measured twice a week after CAR-T treatment and were measured along three orthogonal axes (a, b, and c) and calculated using the equation (a*b*c)/2.

The antitumor effect of CLDN18.2 CARS with YTHDF2 overexpression in CFAPC-1 in vivo was examined, as shown in FIG. 10. Female NOD/SCID/γ−/− (NSG) mice were purchased and maintained. NSG mice (n=6) were subcutaneously inoculated with 2*10\hat{ }6 CFPAC-1. One week after tumor cell inoculation, the mice were randomly grouped and treated with PBS, 1*10\hat{ }7 CLDN18.2 CAR-T or 1*10\hat{ }7 CLDN18.2 CAR-T with YTHDF2 overexpression (CLDN18.2-YTHDF2-OE CAR-T). Tumor volume were measured twice a week after CAR-T treatment and were measured along three orthogonal axes (a, b, and c) and calculated using the equation (a*b*c)/2.

These results indicate that immune cells (e.g., T cells) with attenuated expression/activity of YTHDF2 have enhanced proliferation activity, as well as increased ability to kill tumor cells.

While preferred embodiments of the present application have been shown and described herein, it will be obvious to those skilled in the art that such embodiments are provided by way of example only. It is not intended that the present application be limited by the specific examples provided within the specification. While the present application has been described with reference to the aforementioned specification, the descriptions and illustrations of the embodiments herein are not meant to be construed in a limiting sense. Numerous variations, changes, and substitutions will now occur to those skilled in the art without departing from the present application. Furthermore, it shall be understood that all aspects of the present application are not limited to the specific depictions, configurations or relative proportions set forth herein which depend upon a variety of conditions and variables. It should be understood that various alternatives to the embodiments of the present application described herein may be employed in practicing the present application. It is therefore contemplated that the present application shall also cover any such alternatives, modifications, variations or equivalents. It is intended that the following claims define the scope of the present application and that methods and structures within the scope of these claims and their equivalents be covered thereby.

1. A modified immune cell, which has
attenuated expression and/or activity, relative to an unmodified immune cell, of YTH N6-Methyladenosine RNA Binding Protein 2 (YTHDF2); and
enhanced anti-tumor activity.

2. (canceled)

3. The modified immune cell of claim 1, wherein the immune cell is a T cell.

4-5. (canceled)

6. The modified immune cell of claim 1, wherein the immune cell is a tumor infiltrating T cell, CAR-T cell or a TCR-T cell.

7-8. (canceled)

9. The modified immune cell of claim 1, wherein the unmodified corresponding immune cell is TCF1⁻, Tim3⁺, PD-1⁺, PD-1⁻, TCF1⁺, TCF7⁺ or Tim3⁻.

10-19. (canceled)

20. The modified immune cell of claim 1, wherein the immune cell has been modified with an agent capable of attenuating the expression and/or activity of YTHDF; and
wherein the agent capable of attenuating the expression and/or activity of YTHDF2 comprises at least one selected from the group consisting of: a ubiquitin, a PROTAC, a dsRNA, a siRNA, a shRNA, an aptamer, and a gRNA.

21-22. (canceled)

23. A composition, comprising the modified immune cell of claim 1, and a pharmaceutically acceptable excipient.

24. A composition, comprising the modified immune cell of claim 1, an agent capable of attenuating the expression and/or activity of YTHDF2, and optionally a pharmaceutically acceptable excipient.

25-29. (canceled)

30. The composition of claim 24, wherein the agent capable of attenuating the expression and/or activity of YTHDF2 comprises at least one selected from the group consisting of a ubiquitin, a PROTAC, a dsRNA, a siRNA, a shRNA, an aptamer, and a gRNA.

31. (canceled)

32. The composition of claim 24, further comprising a second active ingredient, wherein the second active ingredient is an anti-cancer agent.

33-34. (canceled)

35. The composition of claim 32, wherein the second active ingredient comprises at least one agent selected from the group consisting of an anti-PD-L1 antibody or an antigen binding portion thereof, an anti-PD-1 antibody or an antigen binding portion thereof, an anti-CTLA-4 antibody or an antigen binding portion thereof, and an 1D0 attenuating agent.

36-37. (canceled)

38. A method for activating an immune cell, comprising:
attenuating an expression and/or activity of YTHDF2 in the immune cell to generate an immune cell having enhanced anti-tumor activity, and/or for preventing and/or reversing exhaustion of an immune cell.

39-41. (canceled)

42. The method of claim 38, wherein the immune cell is a T cell.

43-44. (canceled)

45. The method of claim 38, wherein the immune cell is a tumor infiltrating T cell, a CAR-T cell or a TCR-T cell.

46-52. (canceled)

53. The method of claim 38, wherein the attenuating comprises modifying the immune cell with an agent capable of attenuating the expression and/or activity of YTHDF2 in the immune cell, and
wherein the agent capable of attenuating the expression and/or activity of YTHDF2 comprises at least one selected from the group consisting of a ubiquitin, a PROTAC, dsRNA, a siRNA, a shRNA, an aptamer, and a gRNA.

54-56. (canceled)

57. A method for treating a disease, disorder or condition associated with an expression of a tumor antigen in a subject in need thereof, for treating cancer in a subject in need thereof, for stimulating a T cell-mediated immune response to a cancer cell and/or a tumor antigen in a subject in need thereof, for providing an anti-tumor immunity in a subject in need thereof, and/or for reversing exhaustion of T cells in a subject in need thereof, comprising:
administering to the subject an agent capable of attenuating an expression and/or activity of YTHDF2; and
a modified immune cell of claim 1.

58-75. (canceled)

76. The method of claim 57, wherein the method further comprises:
administering to the subject an additional anti-cancer treatment.

77.-78. (canceled)

79. The method of claim 76, wherein the additional anti-cancer treatment comprises at least one agent selected from the group consisting of an anti-PD-L1 antibody or an antigen binding portion thereof, an anti-PD-1 antibody or an antigen binding portion thereof, an anti-CTLA-4 antibody or an antigen binding portion thereof, and an IDO attenuating agent.

80. (canceled)

81. A method of making a composition comprising of an agent capable of attenuating an expression and/or activity of YTHDF2, comprising:
1) activating an immune cell;
2) generating an immune cell having enhanced anti-tumor activity;
3) preventing and/or reversing exhaustion of an immune cell;
4) treating a disease, disorder or condition associated with an expression of a tumor antigen in a subject in need thereof;
5) treating cancer in a subject in need thereof;
6) stimulating a T cell-mediated immune response to a cancer cell and/or a tumor antigen in a subject in need thereof;
7) providing an anti-tumor immunity in a subject in need thereof,
8) increasing and/or improving proliferation of CD4⁺ T cells;
9) increasing and/or improving proliferation of CD8⁺ T cells;
10) increasing a number of CD8⁺ cytotoxic T cells in or surrounding the site of a tumor;
11) increasing a number of tumor infiltrating CD8⁺ T cells;
12) enhancing cytokine production of T cells;
13) enhancing an antitumor response of a cancer immunotherapy; and
14) reversing exhaustion of T cells in a subject in need thereof.

82-83. (canceled)

84. The method of claim 81, further comprising combining an additional active ingredient in the manufacture of a medicament.

85-86. (canceled)

87. The method of claim 84, wherein the additional active ingredient comprises at least one agent selected from the group consisting of an anti-PD-L1 antibody or an antigen binding portion thereof, an anti-PD-1 antibody or an antigen binding portion thereof, an anti-CTLA-4 antibody or an antigen binding portion thereof, and an IDO attenuating agent.

88. (canceled)


SEQUENCE LISTING (25 sequences)

SEQ ID NO: 1 - DNA, 23 bp, Artificial Sequence
  primer 1
gaacggtattgtcggtattgtca

SEQ ID NO: 2 - DNA, 22 bp, Artificial Sequence
  primer 2
agaccactccaacacagaactt

SEQ ID NO: 3 - DNA, 27 bp, Artificial Sequence
  primer 3
gttctttgtatatattgaatgttagcc

SEQ ID NO: 4 - DNA, 25 bp, Artificial Sequence
  primer 4
tatgctctaaggacaagaattgaca

SEQ ID NO: 5 - DNA, 20 bp, Artificial Sequence
  primer 5
ctttgcagagggctaacagc

SEQ ID NO: 6 - protein, 267 aa, Artificial Sequence
  scFv
AATMETDTLLLWVLLLWVPGSTGTGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 7 - protein, 42 aa, Artificial Sequence
  ISD
KRGRKKLLYIFKQPFMRPVQTTQEEDGCSCRFPEEEEGGCEL

SEQ ID NO: 8 - protein, 112 aa, Artificial Sequence
  CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 45 aa, Artificial Sequence
  hinge
TTTPAPRPPTPAPTIASQPLSLRPEACRPAAGGAVHTRGLDFACD

SEQ ID NO: 10 - protein, 24 aa, Artificial Sequence
  TD
IYIWAPLAGTCGVLLLSLVITLYC

SEQ ID NO: 11 - protein, 493 aa, Artificial Sequence
  20 CAR
AATMETDTLLLWVLLLWVPGSTGTGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 1479 bp, Artificial Sequence
  20 CAR
gccgccaccatggagaccgacaccctcttattatgggtgctgttattatgggtccccggaagcaccggaaccggtcagatcgtgctgagccagagccccgccattctgtccgccagccccggcgagaaggtgaccatgacatgcagagcctcctcctccgtgagctacatccactggttccagcagaagcccggctcctcccctaagccttggatctacgccaccagcaatctggctagcggagtgcccgtgagattcagcggcagcggaagcggaaccagctactctctgaccatcagcagagtggaggccgaggacgccgctacctactactgtcagcagtggaccagcaaccctcctaccttcggcggcggcaccaagctggagatcaagggcggaggaggctccggaggtggaggttctggcggtggaggttcccaagtgcagctgcagcagcccggcgctgagctggtgaaacccggcgcttccgtgaagatgagctgcaaggccagcggctacaccttcaccagctacaacatgcactgggtgaagcagacccccggcagaggactggaatggatcggcgccatttaccccggcaacggcgatacctcctacaaccagaagttcaagggcaaggctacactgaccgccgacaagagcagcagcaccgcctacatgcagctgagctctctgaccagcgaggacagcgccgtgtactactgcgctagaagcacctactacggcggcgactggtacttcaacgtgtggggagccggcacaaccgtgacagtgtccgccgcggccgctacaaccacccccgctcccagacctcctacacccgctcccaccattgccagccagcctctctctttaagacccgaggcttgtaggcccgctgctggaggagccgtgcacacaaggggactggactttgcttgtgatatctatatctgggcccctctggctggaacttgtggagtcctcttattatctttagtgatcactttatactgtaagaggggtcgtaagaagttattatacatcttcaagcagcccttcatgaggcccgtccaaaccacccaagaagaggacggatgtagctgtaggtttcccgaggaggaggagggaggctgcgaattacgtgtcaagttctccagaagcgccgatgcccccgcttaccaacaaggtcagaaccagctgtacaatgagctgaatctgggcagaagagaagagtacgacgtgctggataagaggaggggtcgtgaccccgaaatgggaggcaagcccagaagaaaaaacccccaagaaggactctacaacgagctgcaaaaggataagatggctgaggcctattccgagattggcatgaagggcgagagaaggagaggcaagggccacgacggtttatatcaaggtctctccaccgccaccaaggacacatacgatgctctgcacatgcaagctctgccccccaga

SEQ ID NO: 13 - DNA, 3291 bp, Artificial Sequence
  20-Y CAR
gccgccaccatggagaccgacaccctcttattatgggtgctgttattatgggtccccggaagcaccggaaccggtcagatcgtgctgagccagagccccgccattctgtccgccagccccggcgagaaggtgaccatgacatgcagagcctcctcctccgtgagctacatccactggttccagcagaagcccggctcctcccctaagccttggatctacgccaccagcaatctggctagcggagtgcccgtgagattcagcggcagcggaagcggaaccagctactctctgaccatcagcagagtggaggccgaggacgccgctacctactactgtcagcagtggaccagcaaccctcctaccttcggcggcggcaccaagctggagatcaagggcggaggaggctccggaggtggaggttctggcggtggaggttcccaagtgcagctgcagcagcccggcgctgagctggtgaaacccggcgcttccgtgaagatgagctgcaaggccagcggctacaccttcaccagctacaacatgcactgggtgaagcagacccccggcagaggactggaatggatcggcgccatttaccccggcaacggcgatacctcctacaaccagaagttcaagggcaaggctacactgaccgccgacaagagcagcagcaccgcctacatgcagctgagctctctgaccagcgaggacagcgccgtgtactactgcgctagaagcacctactacggcggcgactggtacttcaacgtgtggggagccggcacaaccgtgacagtgtccgccgcggccgctacaaccacccccgctcccagacctcctacacccgctcccaccattgccagccagcctctctctttaagacccgaggcttgtaggcccgctgctggaggagccgtgcacacaaggggactggactttgcttgtgatatctatatctgggcccctctggctggaacttgtggagtcctcttattatctttagtgatcactttatactgtaagaggggtcgtaagaagttattatacatcttcaagcagcccttcatgaggcccgtccaaaccacccaagaagaggacggatgtagctgtaggtttcccgaggaggaggagggaggctgcgaattacgtgtcaagttctccagaagcgccgatgcccccgcttaccaacaaggtcagaaccagctgtacaatgagctgaatctgggcagaagagaagagtacgacgtgctggataagaggaggggtcgtgaccccgaaatgggaggcaagcccagaagaaaaaacccccaagaaggactctacaacgagctgcaaaaggataagatggctgaggcctattccgagattggcatgaagggcgagagaaggagaggcaagggccacgacggtttatatcaaggtctctccaccgccaccaaggacacatacgatgctctgcacatgcaagctctgccccccagaggctccggcgccacaaacttctctttactgaagcaagctggagacgtggaggagaaccccggtcctcgtacgatgtcggccagcagcctcttggagcagagaccaaaaggtcaaggaaacaaagtacaaaatggatctgtacatcaaaaggatggattaaacgatgatgattttgaaccttacttgagtccacaggcaaggcccaataatgcatatactgccatgtcagattcctacttacccagttactacagtccctccattggcttctcctattctttgggtgaagctgcttggtctacggggggtgacacagccatgccctacttaacttcttatggacagctgagcaacggagagccccacttcctaccagatgcaatgtttgggcaaccaggagccctaggtagcactccatttcttggtcagcatggttttaatttctttcccagtgggattgacttctcagcatggggaaataacagttctcagggacagtctactcagagctctggatatagtagcaattatgcttatgcacctagctccttaggtggagccatgattgatggacagtcagcttttgccaatgagaccctcaataaggctcctggcatgaatactatagaccaagggatggcagcactgaagttgggtagcacagaagttgcaagcaatgttccaaaagttgtaggttctgctgttggtagcgggtccattactagtaacatcgtggcttccaatagtttgcctccagccaccattgctcctccaaaaccagcatcttgggctgatattgctagcaagcctgcaaaacagcaacctaaactgaagaccaagaatggcattgcagggtcaagtcttccgccacccccgataaagcataacatggatattggaacttgggataacaagggtcccgttgcaaaagccccctcacaggctttggttcagaatataggtcagccaacccaggggtctcctcagcctgtaggtcagcaggctaacaatagcccaccagtggctcaggcatcagtagggcaacagacacagccattgcctccacctccaccacagcctgcccagctttcagtccagcaacaggcagctcagccaacccgctgggtagcacctcggaaccgtggcagtgggttcggtcataatggggtggatggtaatggagtaggacagtctcaggctggttctggatctactccttcagaaccccacccagtgttggagaagcttcggtccattaataactataaccccaaagattttgactggaatctgaaacatggccgggttttcatcattaagagctactctgaggacgatattcaccgttccattaagtataatatttggtgcagcacagagcatggtaacaagagactggatgctgcttatcgttccatgaacgggaaaggccccgtttacttacttttcagtgtcaacggcagtggacacttctgtggcgtggcagaaatgaaatctgctgtggactacaacacatgtgcaggtgtgtggtcccaggacaaatggaagggtcgttttgatgtcaggtggatttttgtgaaggacgttcccaatagccaactgcgacacattcgcctagagaacaacgagaataaaccagtgaccaactctagggacactcaggaagtgcctctggaaaaggctaagcaggtgttgaaaattatagccagctacaagcacaccacttccatttttgatgacttctcacactatgagaaacgccaagaggaagaagaaagtgttaaaaaggaacgtcaaggtcgtgggaaataa

SEQ ID NO: 14 - protein, 1096 aa, Artificial Sequence
  20-Y
AATMETDTLLLWVLLLWVPGSTGTGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRTMSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAALKLGSTEVASNVPKVVGSAVGSGSITSNIVASNSLPPATIAPPKPASWADIASKPAKQQPKLKTKNGIAGSSLPPPPIKHNMDIGTWDNKGPVAKAPSQALVQNIGQPTQGSPQPVGQQANNSPPVAQASVGQQTQPLPPPPPQPAQLSVQQQAAQPTRWVAPRNRGSGFGHNGVDGNGVGQSQAGSGSTPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 15 - protein, 579 aa, Artificial Sequence
  Y
MSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAALKLGSTEVASNVPKVVGSAVGSGSITSNIVASNSLPPATIAPPKPASWADIASKPAKQQPKLKTKNGIAGSSLPPPPIKHNMDIGTWDNKGPVAKAPSQALVQNIGQPTQGSPQPVGQQANNSPPVAQASVGQQTQPLPPPPPQPAQLSVQQQAAQPTRWVAPRNRGSGFGHNGVDGNGVGQSQAGSGSTPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 16 - protein, 22 aa, Artificial Sequence
  P2A
GSGATNFSLLKQAGDVEENPGP

SEQ ID NO: 17 - DNA, 20 bp, Artificial Sequence
  target
tgaaccttacttgagtccac

SEQ ID NO: 18 - RNA, 67 nt, Artificial Sequence
  tracrRNA
aaauagcaaguuaaaauaaggcuaguccguuaucaacuugaaaaaguggcaccgagucggugcuuuu

SEQ ID NO: 19 - protein, 21 aa, Artificial Sequence
  T2A
GSGEGRGSLLTCGDVEENPGP

SEQ ID NO: 20 - protein, 23 aa, Artificial Sequence
  E2A
GSGQCTNYALLKLAGDVESNPGP

SEQ ID NO: 21 - protein, 25 aa, Artificial Sequence
  F2A
GSGVKQTLNFDLLKLAGDVESNPGP

SEQ ID NO: 22 - protein, 271 aa, Artificial Sequence
  CLDN18.2 scFv
METDTLLLWVLLLWVPGSTGTGDIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSS

SEQ ID NO: 23 - protein, 69 aa, Artificial Sequence
  CD8 hinge
TTTPAPRPPTPAPTIASQPLSLRPEACRPAAGGAVHTRGLDFACDIYIWAPLAGTCGVLLLSLVITLYC

SEQ ID NO: 24 - DNA, 20 bp, Artificial Sequence
  YTHDF2 sgRNA #5
ctactcacgggccttgcctg

SEQ ID NO: 25 - DNA, 20 bp, Artificial Sequence
  YTHDF2 sgRNA #6
ctgcagagaccaaaaggtca